SEQUENCE LISTING (71 sequences)

SEQ ID NO: 1 - protein, 111 aa, Mus musculus
MSQLAEENPWALSVHEGESVTVNCSYKTSITALQWYRQKSGEGPAQLILIRSNEREKRNGRLRATLDTSSQSSSLSITATRCEDTAVYFCATDPMGYKLTFGTGTSLLVDP

SEQ ID NO: 2 - protein, 115 aa, Mus musculus
MGGIITQTPKFLIGQEGQKLTLKCQQNFNHDTMYWYRQDSGKGLRLIYYSITENDLQKGDLSEGYDASREKKSSFSLTVTSAQKNEMAVFLCASSIRDRGAETLYFGSGTRLTVL

SEQ ID NO: 3 - DNA, 604 bp, Mus musculus
atgagccagctggccgaagaaaatccgtgggccctgagcgtgcatgaaggcgaaagcgtgaccgtgaactgcagctataaaaccagcattaccgcgctgcagtggtatcgtcagaaaagcggcgaaggtccggcgcagctgattctgattcgtagcaacgaacgtgaaaaacgtaacggccgtctgcgtgcgaccctggataccagcagccagagcagcagcctgagcatcaccgcgacccgttgcgaagataccgcggtgtatttttgcgcgaccgatccgatgggctataaactgacctttggcaccggcaccagcctgctggttgatccgaacattcagaatccggaaccggcggtgtatcagctgaaagatccgcgtagccaggatagcaccctgtgcctgtttaccgattttgatagccagattaacgtgccgaaaaccatggaaagcggcacctttattaccgataaaacggtgctggatatgaaagcgatggatagcaaaagcaacggcgcgattgcgtggagcaaccagaccagctttacctgccaggatatcttcaaagaaaccaacgcgacctatccgagcagctaataagctt

SEQ ID NO: 4 - DNA, 733 bp, Mus musculus
atgggcggcattattacccagaccccgaaatttctgattggccaggaaggccagaaactgaccctgaaatgccagcagaactttaaccacgataccatgtattggtatcgtcaggatagcggcaaaggcctgcgtctgatttattatagcatcaccgaaaacgatctgcagaaaggcgatctgagcgaaggctatgatgcgagccgtgaaaaaaaaagcagctttagcctgaccgtgaccagcgcgcagaaaaacgaaatggcggtgtttctgtgcgcgagcagcattcgtgatcgtggcgcggaaaccctgtattttggcagcggcacccgtctgaccgtgctggaagatctgcgtaacgtgaccccgccgaaagtgagcctgtttgaaccgagcaaagcggaaattgcgaacaaacagaaagcgaccctggtgtgcctggcccgtggcttttttccggatcatgtggagctgtcttggtgggtgaacggcaaagaagtgcatagcggcgtttcaaccgatccgcaggcgtataaagaaagcaactatagctatgccctgagcagccgtctgcgtgtgagcgcgaccttttggcataatccgcgtaaccattttcgttgccaggtgcagtttcatggcctgagcgaagaagataaatggccggaaggcagcccgaaaccggtgacccagaacattagcgcggaagcgtggggccgtgcggattaataagctt

SEQ ID NO: 5 - protein, 198 aa, Mus musculus
MSQLAEENPWALSVHEGESVTVNCSYKTSITALQWYRQKSGEGPAQLILIRSNEREKRNGRLRATLDTSSQSSSLSITATRCEDTAVYFCATDPMGYKLTFGTGTSLLVDPNIQNPEPAVYQLKDPRSQDSTLCLFTDFDSQINVPKTMESGTFITDKTVLDMKAMDSKSNGAIAWSNQTSFTCQDIFKETNATYPSS

SEQ ID NO: 6 - protein, 241 aa, Mus musculus
MGGIITQTPKFLIGQEGQKLTLKCQQNFNHDTMYWYRQDSGKGLRLIYYSITENDLQKGDLSEGYDASREKKSSFSLTVTSAQKNEMAVFLCASSIRDRGAETLYFGSGTRLTVLEDLRNVTPPKVSLFEPSKAEIANKQKATLVCLARGFFPDHVELSWWVNGKEVHSGVSTDPQAYKESNYSYALSSRLRVSATFWHNPRNHFRCQVQFHGLSEEDKWPEGSPKPVTQNISAEAWGRAD

SEQ ID NO: 7 - DNA, 604 bp, Artificial Sequence
  Recombinantly produced Mouse T cell receptor
atgagccagctggccgaagaaaatccgtgggccctgagcgtgcatgaaggcgaaagcgtgaccgtgaactgcagctataaaaccagcattaccgcgctgcagtggtatcgtcagaaaagcggcgaaggtccggcgcagctgattctgattcgtagcaacgaacgtgaaaaacgtaacggccgtctgcgtgcgaccctggataccagcagccagagcagcagcctgagcatcaccgcgacccgttgcgaagataccgcggtgtatttttgcgcgaccgatccgatgggctataaactgacctttggcaccggcaccagcctgctggttgatccgaacattcagaatccggaaccggcggtgtatcagctgaaagatccgcgtagccaggatagcaccctgtgcctgtttaccgattttgatagccagattaacgtgccgaaaaccatggaaagcggcacctttattaccgataaatgcgtgctggatatgaaagcgatggatagcaaaagcaacggcgcgattgcgtggagcaaccagaccagctttacctgccaggatatcttcaaagaaaccaacgcgacctatccgagcagctaataagctt

SEQ ID NO: 8 - DNA, 733 bp, Artificial Sequence
  Recombinantly produced Mouse T cell receptor
atgggcggcattattacccagaccccgaaatttctgattggccaggaaggccagaaactgaccctgaaatgccagcagaactttaaccacgataccatgtattggtatcgtcaggatagcggcaaaggcctgcgtctgatttattatagcatcaccgaaaacgatctgcagaaaggcgatctgagcgaaggctatgatgcgagccgtgaaaaaaaaagcagctttagcctgaccgtgaccagcgcgcagaaaaacgaaatggcggtgtttctgtgcgcgagcagcattcgtgatcgtggcgcggaaaccctgtattttggcagcggcacccgtctgaccgtgctggaagatctgcgtaacgtgaccccgccgaaagtgagcctgtttgaaccgagcaaagcggaaattgcgaacaaacagaaagcgaccctggtgtgcctggcccgtggcttttttccggatcatgtggagctgtcttggtgggtgaacggcaaagaagtgcatagcggcgtttgcaccgatccgcaggcgtataaagaaagcaactatagctatgccctgagcagccgtctgcgtgtgagcgcgaccttttggcataatccgcgtaaccattttcgttgccaggtgcagtttcatggcctgagcgaagaagataaatggccggaaggcagcccgaaaccggtgacccagaacattagcgcggaagcgtggggccgtgcggattaataagctt

SEQ ID NO: 9 - protein, 198 aa, Artificial Sequence
  Recombinantly produced Mouse T cell receptor
MSQLAEENPWALSVHEGESVTVNCSYKTSITALQWYRQKSGEGPAQLILIRSNEREKRNGRLRATLDTSSQSSSLSITATRCEDTAVYFCATDPMGYKLTFGTGTSLLVDPNIQNPEPAVYQLKDPRSQDSTLCLFTDFDSQINVPKTMESGTFITDKCVLDMKAMDSKSNGAIAWSNQTSFTCQDIFKETNATYPSS

SEQ ID NO: 10 - protein, 241 aa, Artificial Sequence
  Recombinantly produced Mouse T cell receptor
MGGIITQTPKFLIGQEGQKLTLKCQQNFNHDTMYWYRQDSGKGLRLIYYSITENDLQKGDLSEGYDASREKKSSFSLTVTSAQKNEMAVFLCASSIRDRGAETLYFGSGTRLTVLEDLRNVTPPKVSLFEPSKAEIANKQKATLVCLARGFFPDHVELSWWVNGKEVHSGVCTDPQAYKESNYSYALSSRLRVSATFWHNPRNHFRCQVQFHGLSEEDKWPEGSPKPVTQNISAEAWGRAD

SEQ ID NO: 11 - protein, 111 aa, Artificial Sequence
  Recombinantly produced Mouse T cell receptor
MSQLAEENPWALSVHEGESVTVNCSYKSPMINLQWYRQKSGEGPAQLILIRSNEREKRNGRLRATLDTSSQSSSLSITATRXEDTAVYFCATDPMGYKLTFGTGTSLLVDP

SEQ ID NO: 12 - protein, 111 aa, Artificial Sequence
  Recombinantly produced Mouse T cell receptor
MSQLAEENPWALSVHEGESVTVNCSYKSPMVNLQWYRQKSGEGPAQLILIRSNEREKRNGRLRATLDTSSQSSSLSITATRXEDTAVYFCATDPMGYKLTFGTGTSLLVDP

SEQ ID NO: 13 - protein, 111 aa, Artificial Sequence
  Recombinantly produced Mouse T cell receptor
MSQLAEENPWALSVHEGESVTVNCSYKSLGVNLQWYRQKSGEGPAQLILIRSNEREKRNGRLRATLDTSSQSSSLSITATRXEDTAVYFCATDPMGYKLTFGTGTSLLVDP

SEQ ID NO: 14 - protein, 111 aa, Recombinantly produced Mouse T cell receptor
  MISC_FEATURE (82)..(82): X is cysteine or serine
MSQLAEENPWALSVHEGESVTVNCSYKRMIWTLQWYRQKSGEGPAQLILIRSNEREKRNGRLRATLDTSSQSSSLSITATRXEDTAVYFCATDPMGYKLTFGTGTSLLVDP

SEQ ID NO: 15 - protein, 111 aa, Artificial Sequence
  Recombinantly produced Mouse T cell receptor
MSQLAEENPWALSVHEGESVTVNCSYKGLIWTLQWYRQKSGEGPAQLILIRSNEREKRNGRLRATLDTSSQSSSLSITATRXEDTAVYFCATDPMGYKLTFGTGTSLLVDP

SEQ ID NO: 16 - protein, 111 aa, Artificial Sequence
  Recombinantly produced Mouse T cell receptor
MSQLAEENPWALSVHEGESVTVNCSYKWLGVNLQWYRQKSGEGPAQLILIRSNEREKRNGRLRATLDTSSQSSSLSITATRXEDTAVYFCATDPMGYKLTFGTGTSLLVDP

SEQ ID NO: 17 - protein, 111 aa, Artificial Sequence
  Recombinantly produced Mouse T cell receptor
MSQLAEENPWALSVHEGESVTVNCSYKHLVWTLQWYRQKSGEGPAQLILIRSNEREKRNGRLRATLDTSSQSSSLSITATRXEDTAVYFCATDPMGYKLTFGTGTSLLVDP

SEQ ID NO: 18 - protein, 111 aa, Artificial Sequence
  Recombinantly produced Mouse T cell receptor
MSQLAEENPWALSVHEGESVTVNCSYKSLIWTLQWYRQKSGEGPAQLILIRSNEREKRNGRLRATLDTSSQSSSLSITATRXEDTAVYFCATDPMGYKLTFGTGTSLLVDP

SEQ ID NO: 19 - protein, 111 aa, Artificial Sequence
  Recombinantly produced Mouse T cell receptor
MSQLAEENPWALSVHEGESVTVNCSFSRWFVNLQWYRQKSGEGPAQLILIRSNEREKRNGRLRATLDTSSQSSSLSITATRXEDTAVYFCATDPMGYKLTFGTGTSLLVDP

SEQ ID NO: 20 - protein, 111 aa, Artificial Sequence
  Recombinantly produced Mouse T cell receptor
MSQLAEENPWALSVHEGESVTVNCSYKSPMINLQWYRQKSGEGPAQLILIRSNEREKRNGRLRATLDTSSQSSSLSITATRXEDTAVYFCATDPLGYILTFGTGTSLLVDP

SEQ ID NO: 21 - protein, 111 aa, Artificial Sequence
  Recombinantly produced Mouse T cell receptor
MSQLAEENPWALSVHEGESVTVNCSYKSPMINLQWYRQKSGEGPAQLILIRSNEREKRNGRLRATLDTSSQSSSLSITATRXEDTAVYFCATDPLGYVLTFGTGTSLLVDP

SEQ ID NO: 22 - protein, 111 aa, Artificial Sequence
  Recombinantly produced Mouse T cell receptor
MSQLAEENPWALSVHEGESVTVNCSYKSPMINLQWYRQKSGEGPAQLILIRSNEREKRNGRLRATLDTSSQSSSLSITATRXEDTAVYFCATDPLGYRLTFGTGTSLLVDP

SEQ ID NO: 23 - protein, 111 aa, Artificial Sequence
  Recombinantly produced Mouse T cell receptor
MSQLAEENPWALSVHEGESVTVNCSYKSPMINLQWYRQKSGEGPAQLILIRSNEREKRNGRLRATLDTSSQSSSLSITATRXEDTAVYFCATDPLGWQLTFGTGTSLLVDP

SEQ ID NO: 24 - protein, 111 aa, Artificial Sequence
  Recombinantly produced Mouse T cell receptor
MSQLAEENPWALSVHEGESVTVNCSYKSPMINLQWYRQKSGEGPAQLILIRSNEREKRNGRLRATLDTSSQSSSLSITATRXEDTAVYFCATDPLGWMLTFGTGTSLLVDP

SEQ ID NO: 25 - protein, 111 aa, Artificial Sequence
  Recombinantly produced Mouse T cell receptor
MSQLAEENPWALSVHEGESVTVNCSYKSPMINLQWYRQKSGEGPAQLILIRSNEREKRNGRLRATLDTSSQSSSLSITATRXEDTAVYFCATDPFGWMLTFGTGTSLLVDP

SEQ ID NO: 26 - protein, 115 aa, Artificial Sequence
  Recombinantly produced Mouse T cell receptor
MGGIITQTPKFLIGQEGQKLTLKCQQNFNHDTMYWYRQDSGKGLRLIYYSLTEGHLQKGDLSEGYDASREKKSSFSLTVTSAQKNEMAVFLCASSIRDRGAETLYFGSGTRLTVL

SEQ ID NO: 27 - protein, 115 aa, Artificial Sequence
  Recombinantly produced Mouse T cell receptor
MGGIITQTPKFLIGQEGQKLTLKCQQNFNHDTMYWYRQDSGKGLRLIYYSLTSDTLQKGDLSEGYDASREKKSSFSLTVTSAQKNEMAVFLCASSIRDRGAETLYFGSGTRLTVL

SEQ ID NO: 28 - protein, 115 aa, Artificial Sequence
  Recombinantly produced Mouse T cell receptor
MGGIITQTPKFLIGQEGQKLTLKCQQNFNHDTMYWYRQDSGKGLRLIYYSLTSDHLQKGDLSEGYDASREKKSSFSLTVTSAQKNEMAVFLCASSIRDRGAETLYFGSGTRLTVL

SEQ ID NO: 29 - protein, 115 aa, Artificial Sequence
  Recombinantly produced Mouse T cell receptor
MGGIITQTPKFLIGQEGQKLTLKCQQNFNHDTMYWYRQDSGKGLRLIYYSLTAEHLQKGDLSEGYDASREKKSSFSLTVTSAQKNEMAVFLCASSIRDRGAETLYFGSGTRLTVL

SEQ ID NO: 30 - protein, 115 aa, Artificial Sequence
  Recombinantly produced Mouse T cell receptor
MGGIITQTPKFLIGQEGQKLTLKCQQNFNHDTMYWYRQDSGKGLRLIYYSLLAGHLQKGDLSEGYDASREKKSSFSLTVTSAQKNEMAVFLCASSIRDRGAETLYFGSGTRLTVL

SEQ ID NO: 31 - protein, 115 aa, Artificial Sequence
  Recombinantly produced Mouse T cell receptor
MGGIITQTPKFLIGQEGQKLTLKCQQNFNHDTMYWYRQDSGKGLRLIYYSLDLVHLQKGDLSEGYDASREKKSSFSLTVTSAQKNEMAVFLCASSIRDRGAETLYFGSGTRLTVL

SEQ ID NO: 32 - protein, 115 aa, Artificial Sequence
  Recombinantly produced Mouse T cell receptor
MGGIITQTPKFLIGQEGQKLTLKCQQNFNHDTMYWYRQDSGKGLRLIYYSLDRSHLQKGDLSEGYDASREKKSSFSLTVTSAQKNEMAVFLCASSIRDRGAETLYFGSGTRLTVL

SEQ ID NO: 33 - protein, 115 aa, Artificial Sequence
  Recombinantly produced Mouse T cell receptor
MGGIITQTPKFLIGQEGQKLTLKCQQNFNHDTMYWYRQDSGKGLRLIYYSIDMGHLQKGDLSEGYDASREKKSSFSLTVTSAQKNEMAVFLCASSIRDRGAETLYFGSGTRLTVL

SEQ ID NO: 34 - protein, 115 aa, Artificial Sequence
  Recombinantly produced Mouse T cell receptor
MGGIITQTPKFLIGQEGQKLTLKCQQNFNHDTMYWYRQDSGKGLRLIYYSLARSHLQKGDLSEGYDASREKKSSFSLTVTSAHKNEMAVFLCASSIRDRGAETLYFGSGTRLTVL

SEQ ID NO: 35 - protein, 115 aa, Artificial Sequence
  Recombinantly produced Mouse T cell receptor
MGGIITQTPKFLIGQEGQKLTLKCQQNFNHDTMYWYRQDSGKGLRLIYYSLARSHLQKGDLSEGYDASREKKSSFSLTVTSAQKNEMAVFLCASSIRDRGAETLYFGSGTRLTVL

SEQ ID NO: 36 - protein, 115 aa, Artificial Sequence
  Recombinantly produced Mouse T cell receptor
MGGIITQTPKFLIGQEGQKLTLKCQQNFNHDTMYWYRQDSGKGLRLIYYSINARHLQKGDLSEGYDASREKKSSFSLTVTSAQKNEMAVFLCASSIRDRGAETLYFGSGTRLTVL

SEQ ID NO: 37 - protein, 115 aa, Artificial Sequence
  Recombinantly produced Mouse T cell receptor
MGGIITQTPKFLIGQEGQKLTLKCQQNFNHDTMYWYRQDSGKGLRLIYYSIDSHHLQKGDLSEGYDASREKKSSFSLTVTSAQKNEMAVFLCASSIRDRGAETLYFGSGTRLTVL

SEQ ID NO: 38 - protein, 115 aa, Artificial Sequence
  Recombinantly produced Mouse T cell receptor
MGGIITQTPKFLIGQEGQKLTLKCQQNFNHDTMYWYRQDSGKGLRLIYYSITPTHLQKGDLSEGYDASREKKSSFSLTVTSAQKNEMAVFLCASSIRDRGAETLYFGSGTRLTVL

SEQ ID NO: 39 - protein, 115 aa, Artificial Sequence
  Recombinantly produced Mouse T cell receptor
MGGIITQTPKFLIGQEGQKLTLKCQQNFNHDTMYWYRQDSGKGLRLIYYSITTNHLQKGDLSEGYDASREKKSSFSLTVTSAQKNEMAVFLCASSIRDRGAETLYFGSGTRLTVL

SEQ ID NO: 40 - protein, 115 aa, Artificial Sequence
  Recombinantly produced Mouse T cell receptor
MGGIITQTPKFLIGQEGQKLTLKCQQNFNHDTMYWYRQDSGKGLRLIYYSITENDLQKGDLSEGYDASREKKSSFSLTVTSTQKNEMAVFLCASSKRKNGAETLYFGSGTRLTVL

SEQ ID NO: 41 - protein, 115 aa, Artificial Sequence
  Recombinantly produced Mouse T cell receptor
MGGIITQTPKFLIGQEGQKLTLKCQQNFNHDTMYWYRQDSGKGLRLIYYSITENDLQKGDLSEGYDASREKKSSFSLTVTSTQKNEMAVFLCASSKRENGAETLYFGSGTRLTVL

SEQ ID NO: 42 - protein, 115 aa, Artificial Sequence
  Recombinantly produced Mouse T cell receptor
MGGIITQTPKFLIGQEGQKLTLKCQQNFNHDTMYWYRQDSGKGLRLIYYSITENDLQKGDLSEGYDASREKKSSFSLTVTSTQKNEMAVFLCASSRRENGAETLYFGSGTRLTVL

SEQ ID NO: 43 - protein, 115 aa, Artificial Sequence
  Recombinantly produced Mouse T cell receptor
MGGIITQTPKFLIGQEGQKLTLKCQQNFNHDTMYWYRQDSGKGLRLIYYSITENDLQKGDLSEGYDASREKKSSFSLTVTSPQKNEMAVFLCASSKRRNGAETLYFGSGTRLTVL

SEQ ID NO: 44 - protein, 115 aa, Artificial Sequence
  Recombinantly produced Mouse T cell receptor
MGGIITQTPKFLIGQEGQKLTLKCQQNFNHDTMYWYRQDSGKGLRLIYYSITENDLQKGDLSEGYDASREKKSSFSLTVTSTQKNEMAVFLCASSKRRDGAETLYFGSGTRLTVL

SEQ ID NO: 45 - protein, 115 aa, Artificial Sequence
  Recombinantly produced Mouse T cell receptor
MGGIITQTPKFLIGQEGQKLTLKCQQNFNHDTMYWYRQDSGKGLRLIYYSITENDLQKGDLSEGYDASREKKSSFSLTVTSTQKNEMAVFLCASSNRRNGAETLYFGSGTRLTVL

SEQ ID NO: 46 - protein, 115 aa, Artificial Sequence
  Recombinantly produced Mouse T cell receptor
MGGIITQTPKFLIGQEGQKLTLKCQQNFNHDTMYWYRQDSGKGLRLIYYSITENDLQKGDLSEGYDASREKKSSFSLTVTSVQKNEMAVFLCASSRRREGAETLYFGSGTRLTVL

SEQ ID NO: 47 - protein, 115 aa, Artificial Sequence
  Recombinantly produced Mouse T cell receptor
MGGIITQTPKFLIGQEGQKLTLKCQQNFNHDTMYWYRQDSGKGLRLIYYSITENDLQKGDLSEGYDASREKKSSFSLTVTSTQKNEMAVFLCASSKRLRGAETLYFGSGTRLTVL

SEQ ID NO: 48 - protein, 115 aa, Artificial Sequence
  Recombinantly produced Mouse T cell receptor
MGGIITQTPKFLIGQEGQKLTLKCQQNFNHDTMYWYRQDSGKGLRLIYYSITENDLQKGDLSEGYDASREKKSSFSLTVTSAQKNEMAVFLCASSVRRFGAETLYFGSGTRLTVL

SEQ ID NO: 49 - protein, 115 aa, Artificial Sequence
  Recombinantly produced Mouse T cell receptor
MGGIITQTPKFLIGQEGQKLTLKCQQNFNHDTMYWYRQDSGKGLRLIYYSITENDLQKGDLSEGYDASREKKSSFSLTVTSAQKNEMAVFLCASSFRRHGAETLYFGSGTRLTVL

SEQ ID NO: 50 - protein, 115 aa, Artificial Sequence
  Recombinantly produced Mouse T cell receptor
MGGIITQTPKFLIGQEGQKLTLKCQQNFNHDTMYWYRQDSGKGLRLIYYSITENDLQKGDLSEGYDASREKKSSFSLTVTSAQKNEMAVFLCASSIRDRGAEKYFFGSGTRLTVL

SEQ ID NO: 51 - protein, 115 aa, Artificial Sequence
  Recombinantly produced Mouse T cell receptor
MGGIITQTPKFLIGQEGQKLTLKCQQNFNHDTMYWYRQDSGKGLRLIYYSITENDLQKGDLSEGYDASREKKSSFSLTVTSAQKNEMAVFLCASSIRDRGAERLIFGSGTRLTVL

SEQ ID NO: 52 - protein, 115 aa, Artificial Sequence
  Recombinantly produced Mouse T cell receptor
MGGIITQTPKFLIGQEGQKLTLKCQQNFNHDTMYWYRQDSGKGLRLIYYSLLAGHLQKGDLSEGYDASREKKSSFSLTVTSAQKNEMAVFLCASSKRKNGAETLYFGSGTRLTVL

SEQ ID NO: 53 - protein, 115 aa, Artificial Sequence
  Recombinantly produced Mouse T cell receptor
MGGIITQTPKFLIGQEGQKLTLKCQQNFNHDTMYWYRQDSGKGLRLIYYSLLAGHLQKGDLSEGYDASREKKSSFSLTVTSAQKNEMAVFLCASSNRRNGAETLYFGSGTRLTVL

SEQ ID NO: 54 - protein, 115 aa, Artificial Sequence
  Recombinantly produced Mouse T cell receptor
MGGIITQTPKFLIGQEGQKLTLKCQQNFNHDTMYWYRQDSGKGLRLIYYSLDRSHLQKGDLSEGYDASREKKSSFSLTVTSAQKNEMAVFLCASSKRKNGAETLYFGSGTRLTVL

SEQ ID NO: 55 - protein, 115 aa, Artificial Sequence
  Recombinantly produced Mouse T cell receptor
MGGIITQTPKFLIGQEGQKLTLKCQQNFNHDTMYWYRQDSGKGLRLIYYSLDRSHLQKGDLSEGYDASREKKSSFSLTVTSAQKNEMAVFLCASSNRRNGAETLYFGSGTRLTVL

SEQ ID NO: 56 - protein, 90 aa, Artificial Sequence
  Recombinantly produced Mouse T cell receptor
XIQNPEPAVYQLKDPRSQDSTLCLFTDFDSQINVPKTMESGTFITDKTVLDMKAMDSKSNGAIAWSNQTSFTCQDIFKETNATYPSSDVP

SEQ ID NO: 57 - protein, 126 aa, Artificial Sequence
  Recombinantly produced Mouse T cell receptor
EDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRAD

SEQ ID NO: 58 - DNA, 3342 bp, Artificial Sequence
  Plasmid pEX954
gatctcgatcccgcgaaattaatacgactcactatagggagaccacaacggtttccctctagaaataattttgtttaactttaagaaggagatataatcgatgtctaactcgagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctaagcttgaattccgatccggctgctaacaaagcccgaaaggaagctgagttggctgctgccaccgctgagcaataactagcataaccccttggggcctctaaacgggtcttgaggggttttttgctgaaaggaggaactatatccggataattcttgaagacgaaagggcctcgtgatacgcctatttttataggttaatgtcatgataataatggtttcttagacgtgaggtggcacttttcggggaaatgtgcgcggaacccctatttgtttatttttctaaatacattcaaatatgtatccgctcatgagacaataaccctgataaatgcttcaataatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcag

SEQ ID NO: 59 - DNA, 3836 bp, Artificial Sequence
  Plasmid pEX821
gatctcgatcccgcgaaattaatacgactcactatagggagaccacaacggtttccctctagaaataattttgtttaactttaagaaggagatatacatatgaacgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcaggccgggactagcgggagggcgaccagagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccaccggtttctaccccgaccacgtggagctgagctggtgggtgaatgggaaggaggtgcacagtggggtctgcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagactaagcttgaattccgatccggctgctaacaaagcccgaaaggaagctgagttggctgctgccaccgctgagcaataactagcataaccccttggggcctctaaacgggtcttgaggggttttttgctgaaaggaggaactatatccggataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcaatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcag

SEQ ID NO: 60 - protein, 205 aa, Artificial Sequence
  Recombinantly produced Mouse T cell receptor
MSQLAEENPWALSVHEGESVTVNCSYKSPMINLQWYRQKSGEGPAQLILIRSNEREKRNGRLRATLDTSSQSSSLSITATRSEDTAVYFCATDPLGYILTFGTGTSLLVDPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESS

SEQ ID NO: 61 - protein, 245 aa, Artificial Sequence
  Recombinantly produced Mouse T cell receptor
MGGIITQTPKFLIGQEGQKLTLKCQQNFNHDTMYWYRQDSGKGLRLIYYSLLAGHLQKGDLSEGYDASREKKSSFSLTVTSTQKNEMAVFLCASSKRKNGAETLYFGSGTRLTVLEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 62 - protein, 9 aa, Mus musculus
LYLVCGERG

SEQ ID NO: 63 - protein, 30 aa, Artificial Sequence
  Synthetic linker sequence
PGGGSGGGGSGGGGSGGGGSGGGGSGGGGP

SEQ ID NO: 64 - protein, 35 aa, Artificial Sequence
  Synthetic linker sequence
PGGGSGGGGSGGGGSGGGGSGGGGSGGGGSGGGGP

SEQ ID NO: 65 - protein, 9 aa, Artificial Sequence
  Synthetic mutant murine peptide
LYLVCGERL

SEQ ID NO: 66 - protein, 94 aa, Homo sapiens
NIQNPDPAVYQLRDSKSSDKSVCLFTDFDSQTNVSQSKDSDVYITDKTVLDMRSMDFKSNSAVAWSNKSDFACANAFNNSIIPEDTFFPSPESS

SEQ ID NO: 67 - protein, 130 aa, Homo sapiens
EDLNKVFPPEVAVFEPSEAEISHTQKATLVCLATGFFPDHVELSWWVNGKEVHSGVSTDPQPLKEQPALNDSRYALSSRLRVSATFWQDPRNHFRCQVQFYGLSENDEWTQDRAKPVTQIVSAEAWGRAD

SEQ ID NO: 68 - protein, 130 aa, Homo sapiens
EDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRAD

SEQ ID NO: 69 - protein, 372 aa, Artificial Sequence
  Recombinantly produced Mouse T cell receptor
MHIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYSGGEGGGPGGIITQTPKFLIGQEGQKLTLKCQQNFNHDTMYWYRQDSGKGLRLIYYSLLAGHLQKGDLSEGYDASREKKSSFSLTVTSTQKNEMAVFLCASSKRKNGAETLYFGSGTRLTVLEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 70 - protein, 363 aa, Artificial Sequence
  Recombinantly produced Mouse T cell receptor
MGPVPRSVSLPLTLKELIEELSNITQDQTPLCNGSMVWSVDLAAGGFCVALDSLTNISNCNAIYRTQRILHGLCNRKAPTTVSSLPDTKIEVAHFITKLLSYTKQLFRHGPFGGEGGGPGGIITQTPKFLIGQEGQKLTLKCQQNFNHDTMYWYRQDSGKGLRLIYYSLLAGHLQKGDLSEGYDASREKKSSFSLTVTSTQKNEMAVFLCASSKRKNGAETLYFGSGTRLTVLEDLKNVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 71 - protein, 9 aa, Mus musculus
VYLKTNVFL

(19) United States
(12) Patent Application Publication
(10) Pub. No.: US 2012/0027683 A1
(43) Pub. Date: Feb. 2, 2012

(54) DIABETES T CELL RECEPTORS

(76) Inventors: Neill MacKenzie, St. Albans (GB); Bent Jakobsen, Oxfordshire (GB); Daniel Williams, Oxfordshire (GB); Brian Cameron, Oxfordshire (GB)

(21) Appl. No.: 13/060,230

(22) PCT Filed: Sep. 2, 2009
(86) PCT No.: PCT/GB09/02119

§ 371 Date: Oct. 11, 2011

(30) Foreign Application Priority Data
Sep. 4, 2008 (GB) 0816096.2

(51) Int. Cl. A61K 49/00; A61P 3/10; C12N 5/10; C12N 1/21; C07K 14/725; A61K 38/17

(52) U.S. Cl. 424/9.2; 530/350; 530/402; 530/351; 530/409; 424/9.1; 514/1.1; 435/325; 435/252.33; 435/352

(57) ABSTRACT

T cell receptors (TCRs) having the property of binding to a murine insulin-derived peptide, LYLVCGERG (SEQ ID NO:62), presented by the murine H-2K^{d} complex (LYLVCGERG-H-2K^{d}). The TCRs comprise at least one TCR α chain variable domain and/or at least one TCR β chain variable domain and have a K_{D} for the LYLVCGERG-H-2K^{d} complex of less than or equal to 1 μM and/or have an off-rate (k_{off}) for the LYLVCGERG-H-2K^{d} complex of 0.5 S⁻¹ or slower, and use of such TCRs as research tools.

The present invention relates to islet cell-specific T cell receptors (TCRs) and uses thereof. The invention also provides T cell receptors (TCRs) having the property of binding to a murine insulin-derived peptide, LYLVCGERG (SEQ ID NO: 62), presented by the murine H-2K^{d} complex (LYLVCGERG-H-2K^{d}). The TCRs comprise at least one TCR α chain variable domain and/or at least one TCR β chain variable domain and have a K_{D} for the said LYLVCGERG-H-2K^{d} complex of less than or equal to 1 μM and/or have an off-rate (k_{off}) for the LYLVCGERG-H-2K^{d} complex of 0.5 S⁻¹ or slower.

### BACKGROUND TO THE INVENTION

Type 1 diabetes mellitus (T1DM) is an auto-immune disease characterised by metabolic dysfunction, most notably dysregulation of glucose metabolism, accompanied by characteristic long-teen vascular and neurological complications. T1DM is one of the commonest autoimmune diseases, affecting one in 250 individuals in the US where there are approximately 10,000 to 15,000 new cases reported each year, and the incidence is rising. The highest prevalence of T1DM is found in northern Europe, where more than 1 in every 150 Finns develops T1DM by the age of 15. In contrast, T1DM is less common in black and Asian populations where the frequency is less than half that among the white population.

T1DM is characterised by absolute insulin deficiency, making patients dependent on exogenous insulin for survival. Prior to the acute clinical onset of T1DM with symptoms of hyperglycaemia there is a long asymptomatic preclinical period, during which insulin-producing beta cells are progressively destroyed. The autoimmune destruction of beta cells (β cells) is associated with lymphocytic infiltration. In addition, abnormalities in the presentation of MHC Class I antigens on the cell surface have been identified in both animal models and in human T1DM. This immune abnormality may explain why humans become intolerant of self-antigens although it is not clear why only beta cells are preferentially destroyed.

There is a need for new means of identifying and validating compositions for the treatment of T1DM, which the substances and methods described herein will address.

There is ample evidence that CD8 cells are involved in the disease process that leads to T1DM. Histological analysis of the islets in an affected individual shows infiltration by CD8 T cells. In animal models of T1DM, the disease process may be transferred from a diseased animal to a healthy animal using CD8⁺ T cells. There is a genetic association between the development of T1DM and certain HLA class I molecules that are critical for CD8 target recognition. Finally, activated CD8 T cells are present in the circulation of high-risk subjects who develop T1DM.

The LYLVCGERG (SEQ ID NO: 62) peptide is derived from the beta-chain of murine insulin. The peptide is loaded by the murine H-2K^{d} complex and presented on the surface of insulin-producing beta-cells. Therefore, the LYLVCGERG-H-2K^{d} complex provides a murine beta cell-specific marker that TCRs can target, for example for the purpose of delivering therapeutic agents to said beta cells in order to investigate their efficacy. However, for that purpose it would be desirable if the TCR had a high affinity and/or a slow off-rate for the peptide-HLA complex.

### DETAILED DESCRIPTION OF THE INVENTION

The first aspect of the invention provides a method of assessing the efficacy of a putative anti-diabetic agent in a mammalian model of diabetes comprising:

treating one or a plurality of the said model mammals with a high affinity beta cell specific TCR associated with the said agent,
monitoring the effect of such treatment on the treated mammals,
comparing the said effect with
(i) the null effect in one or more untreated control model mammals; and/or
(ii) the effect of treatment of one or more model mammals with the said agent in a form not associated with the said TCR; and/or
(iii) the effect of treatment of one or more model mammals with a different anti-diabetic or putative anti-diabetic agent in a form associated or not associated with the said TCR and/or
(iv) the effect of treatment of one or more model mammals with the said agent in a form associated with an irrelevant TCR.

The effect of treatment of the model mammals may be assessed by any suitable method known to those skilled in the art. The model mammal may be any suitable species, such as a mouse.

Specific embodiments of the present aspect are provided by a method of assessing the efficacy of a putative anti-diabetic agent in a murine model of diabetes comprising:

treating one or a plurality of the said model mice with a TCR associated with the said agent, wherein the TCR is a high affinity murine insulin specific TCR
monitoring the effect of such treatment on the treated mice,
comparing the said effect with
(i) the null effect in one or more untreated control model mice; and/or
(ii) the effect of treatment of one or more model mice with the said agent in a form not associated with the said TCR; and/or
(iii) the effect of treatment of one or more model mice with the a different anti-diabetic or putative anti-diabetic agent in a form associated or not associated with the said TCR and/or
(iv) the effect of treatment of one or more model mice with the said agent in a form associated with an irrelevant TCR.

A second aspect of the invention provides a method of assessing the mass of islet cells present in a mammal, said method comprising:
(i) contacting islet cells of the mammal with a high affinity islet cell specific TCR associated with a detectable moiety,
(ii) detecting a signal from the islet cell-bound TCRs associated with a detectable label, and
(iii) using said signal to estimate the mass of islet cells present.

This method may be carried out at a number of different time points in order to estimate changes in islet cell mass that occur. For example, the method may be carried out at the start and at the end of a clinical trial. Optionally, said method may also be carried out at additional time points during a clinical trial.

Specific embodiments of the present aspect are provided by a method of assessing the mass of islet cells present in a mouse, said method comprising.
(i) contacting islet cells of the mouse with a high affinity murine insulin specific TCR associated with a detectable moiety,
(ii) detecting a signal from the islet cell-bound TCRs associated with a detectable label, and
(iii) using said signal to estimate the mass of islet cells present.

The detectable moiety associated with the said islet cell specific TCRs may be any suitable moiety known to those skilled in the art. For example, said moiety may be an enzyme, a dye (such as a dye which provides an infra-red signal), an MRI-detectable reagent or a radionuclide. The mammal may be any suitable species, such as a mouse.

A third aspect of the present invention is provided by TCRs suitable for use in the methods of the first and second aspects. As such TCRs are being used as targeting agents it would be beneficial if they had a high affinity (K_{D}) and/or a slow off-rate (k_{off}) for the interaction with the islet cell-specific peptide-MHC complexes to which they bind. The preferred method for generating high affinity variants of islet cell specific TCRs for use in the methods of the first and second aspect of the present invention is selection from a diverse library of phage particles displaying such TCRs as disclosed in WO 2004/044004.

The islet cell-specific TCRs of the invention may be derived from any mammal, for example said TCRs may be human or murine TCRs. Furthermore, said islet cell-specific TCRs may be specific for any islet cell specific peptide-MHC. For example said TCRs may be specific for an insulin or IGRP-derived peptide presented in the context of a MHC molecule.

Certain embodiments of the present aspect are provided by a T-cell receptor (TCR) having the property of binding to the LYLVCGERG-H-2K^{d} complex and comprising at least one TCR α chain variable domain and/or at least one TCR β chain variable domain CHARACTERISED IN THAT said TCR has a K_{D} for the said LYLVCGERG-H-2K^{d} complex of less than or equal to 1 μM and/or has an off-rate (k_{off}) for the LYLVCGERG-H-2K^{d} complex of 0.5 S⁻¹ or slower. The K_{D} and/or (k_{off}) measurement can be made by any of the known methods. A preferred method is the Surface Plasmon Resonance (Biacore) method of Example 4.

Preferably, said TCRs have a K_{D} for the said LYLVCGERG-H-2K^{d} complex of less than or equal to 0.1 μM and/or have an off-rate (k_{off}) for the LYLVCGERG-H-2K^{d} complex of 0.1 S⁻¹ or slower.

More preferably, said TCRs have a K_{D} for the said LYLVCGERG-H-2K^{d} complex of less than or equal to 0.01 μM and/or have an off-rate (k_{off}) for the LYLVCGERG-H-2K^{d} complex of 1×10⁻³ S⁻¹ or slower.

Most preferably, said TCRs have a K_{D} for the said LYLVCGERG-H-2K^{d} complex of less than or equal to 0.001 μM and/or have an off-rate (k_{off}) for the LYLVCGERG-H-2K^{d} complex of 1×10⁻⁴ S⁻¹ or slower.

For comparison, the interaction of a disulfide-linked soluble variant of wild-type murine TCR (see SEQ ID NO: 9 for TCR α chain and SEQ ID NO: 10 for TCR chain) and the LYLVCGERG-H-2K^{d} complex has a K_{D} of approximately 2.6 μM and an off-rate (k_{off}) of approximately 0.69 S⁻¹ as measured by the Biacore-base method of Example 4. The off-rate (k_{off}) value determined for the interaction between this wild-type TCR and the LYLVCGERG-H-2K^{d} complex may vary slightly between repeat assays. However, the off-rate (k_{off}) for this interaction is higher (i.e. faster) than 0.5 S⁻¹.

The wild-type TCR was originally isolated from T cells obtained from the islet cells of young non-obese diabetic (NOD) mice. (Wong et al., (1996) J Exp Med. 183 (1); 67-76). This wild-type murine TCR specific for the LYLVCGERG-H-2K^{d} complex has the following Valpha chain and Vbeta chain gene usage:
Alpha chain—AV8-1
Beta chain—BV-19

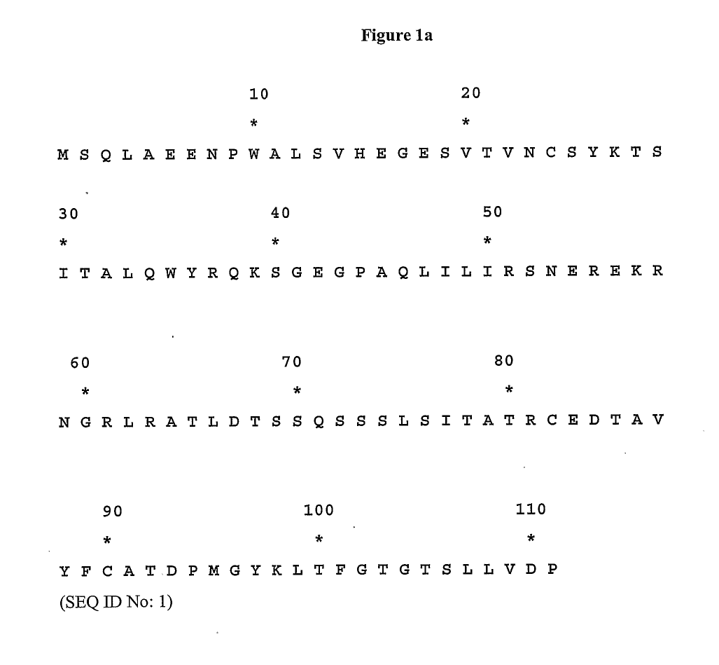

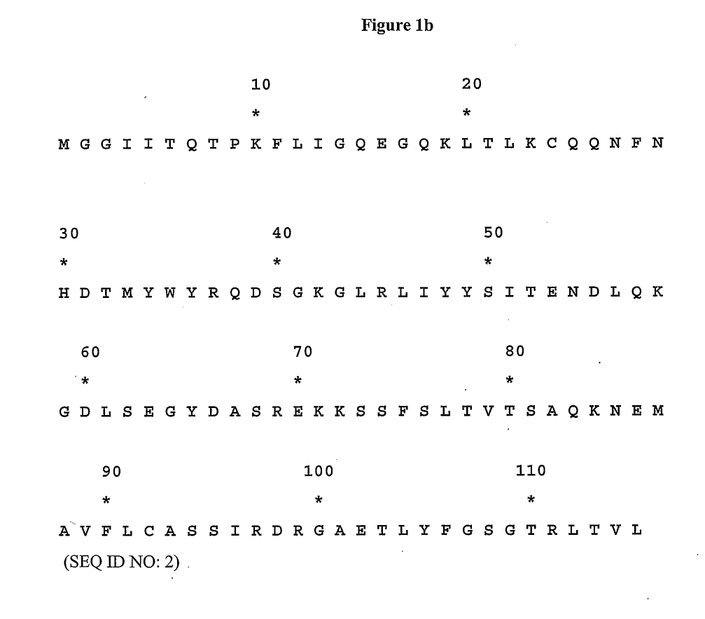

The wild-type murine TCR specific for the LYLVCGERG-H-2K^{d} complex can be used as a template from which other TCRs of the invention with high affinity and/or a slow off-rate for the interaction between said TCRs and the LYLVCGERG-H-2K^{d} complex can be produced. Thus the invention includes TCRs which are mutated relative to the wild-type murine TCR α chain variable domain (see FIG. 1a and SEQ ID No: 1) and/or β chain variable domain (see FIG. 1b and SEQ ID NO: 2) in at least one complementarity determining region (CDR) and/or variable domain framework region thereof.

Phage display provides one means by which libraries of TCR variants can be generated. Methods suitable for the phage display and subsequent screening of libraries of TCR variants each containing a non-native disulfide interchain bond are detailed in (Li et al., (2005) Nature Biotech 23 (3): 349-354) and WO 2004/04404.

Native TCRs exist in heterodimeric αβ or γδ forms. However, recombinant TCRs consisting of a single TCR α or TCR β chain have previously been shown to bind to peptide MHC molecules. Furthermore, recombinant TCRs consisting of αα or ββ homodimers have previously been shown to bind to peptide MHC molecules. Therefore, one embodiment of the invention is provided by TCR αα or TCR ββ homodimers.

In one embodiment the TCR of the invention comprise both a TCR α chain variable domain and a TCR β chain variable domain.

As will be obvious to those skilled in the art the mutation(s) in the TCR α chain sequence and/or TCR β chain sequence may be one or more of substitution(s), deletion(s) or insertion(s). These mutations can be carried out using any appropriate method including, but not limited to, those based on polymerase chain reaction (PCR), restriction enzyme-based cloning, or ligation independent cloning (LIC) procedures. These methods are detailed in many of the standard molecular biology texts. For further details regarding polymerase chain reaction (PCR) mutagenesis and restriction enzyme-based cloning see (Sambrook & Russell, (2001) Molecular Cloning—A Laboratory Manual (3^{rd} Ed.) CSHL Press) Further information on LIC procedures can be found in (Rashtchian, (1995) Curr Opin Biotechnol 6 (1): 30-6)

It should be noted that any αβ TCR that comprises similar Valpha and Vbeta gene usage and therefore amino acid sequence to that of the wild-type murine TCR could make a convenient template TCR. It would then be possible to introduce into the DNA encoding one or both of the variable domains of the template αβ TCR the changes required to produce the mutated high affinity TCRs of the invention. As will be obvious to those skilled in the art, the necessary mutations could be introduced by a number of methods, for example site-directed mutagenesis.

The TCRs of the invention include those in which one or more of the TCR alpha chain and/or beta chain variable region amino acids corresponding to those listed below are mutated relative to the amino acid occurring at these positions in the sequences provided for the wild-type murine TCR alpha chain variable region in FIG. 1a and SEQ ID No: 1 and for the wild-type murine TCR beta chain variable region in FIG. 1b and SEQ ID No: 2.

Unless stated to the contrary, the TCR amino acid sequences herein are generally provided including an N-terminal methionine (Met or M) residue. As will be known to those skilled in the art this residue may be removed during the production of recombinant proteins. As will also be obvious to those skilled in the art, it may be possible to truncate the sequences provided at the C-terminus and/or N-terminus thereof, by 1, 2, 3, 4, 5 or more residues, without substantially affecting the peptide-MHC (pMHC) binding characteristics of the TCR, all such trivial variants are encompassed by the present invention.

As used herein the term “variable region” is understood to encompass the amino acid sequences of a given TCR which are encoded by the murine or human TRAV and TRAJ genes for TCR α chains and either the murine or human TRBV, TRBJ and TRBD genes for TCR β chains.

As used herein the term “variable domain” is understood to encompass the amino acid sequences of a given TCR which are encoded by a murine or human TRAV gene for TCR α chains and a TRBV gene for murine or human TCR β chains. (T cell receptor Factsbook, (2001) LeFranc and LeFranc, Academic Press, ISBN 0-12-441352-8)

Embodiments of the invention also include TCRs which comprise mutation of one or more of the TCR alpha chain variable region amino acids corresponding to those listed below, relative to the amino acid occurring at these positions in the sequence provided for the TCR alpha chain variable region of the wild-type murine TCR alpha chain in FIG. 1a and SEQ ID No: 1. The amino acids referred to which may be mutated are: 26Y, 27K, 28T, 29S, 30I, 31T, 32A, 95M, 97Y and 98K for example the amino acids:
26F
27S
28S, R, G, W or H
29P, L, M, or W
30M, G, V or F
31I, V or W
32N or T
95L or F
97W
98I, V, R, Q or M.

The numbering used above is the same as that shown in FIG. 1a and SEQ ID No: 1

Embodiments of the invention also include TCRs which comprise mutation of one or more of the TCR beta chain variable region amino acids corresponding to those listed below, relative to the amino acid occurring at these positions in the sequence provided for the TCR beta chain variable region of the wild-type murine TCR beta chain in FIG. 1b and SEQ ID No: 2. The amino acids referred to which may be mutated are: 51I, 52T, 53E, 54N, 55D, 82A, 83Q, 96I, 98D, 99R, 103T, 104L and 105Y, for example:

### 51L

### 52L, D, A or N

### 53S, A, L, R, M, P or T

### 54G, D, E, V, S, R, H or T

### 55H or T

### 82T, P or V

### 83H

### 96K, R, N, V or F

### 98K, E, R or L

### 99N, D, E, F or H

### 103K or R

### 104Y

### 105F or I

The numbering used above is the same as that shown in FIG. 1b and SEQ ID No: 2

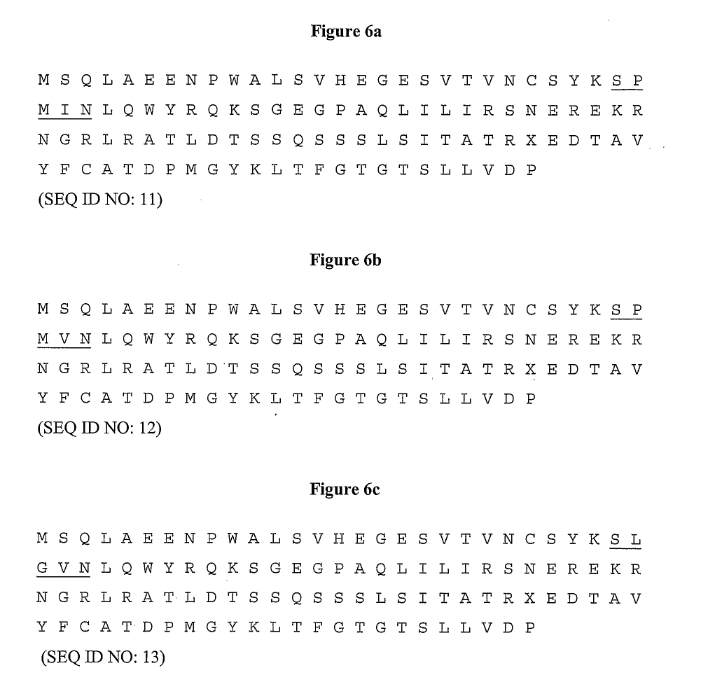

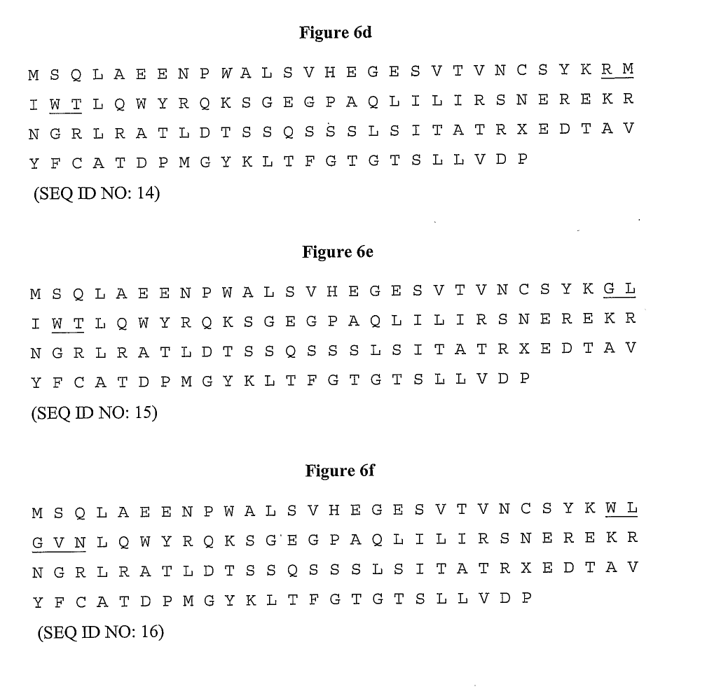

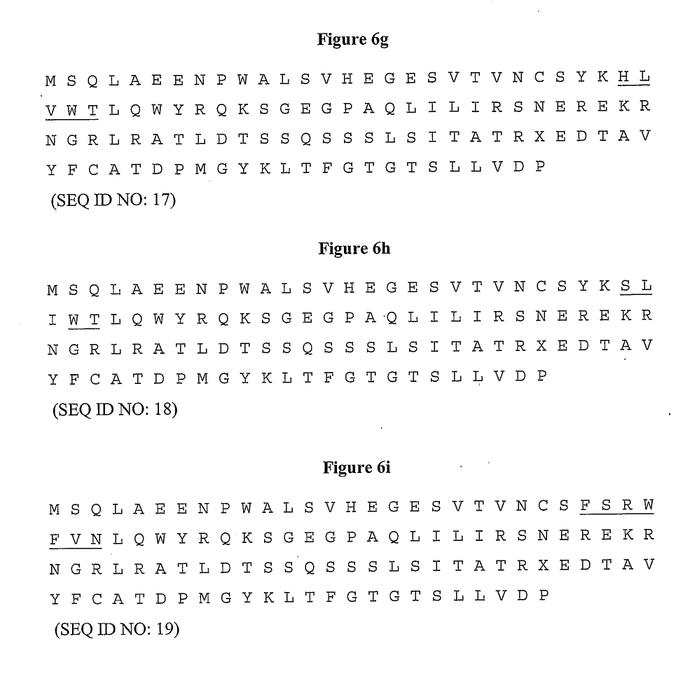

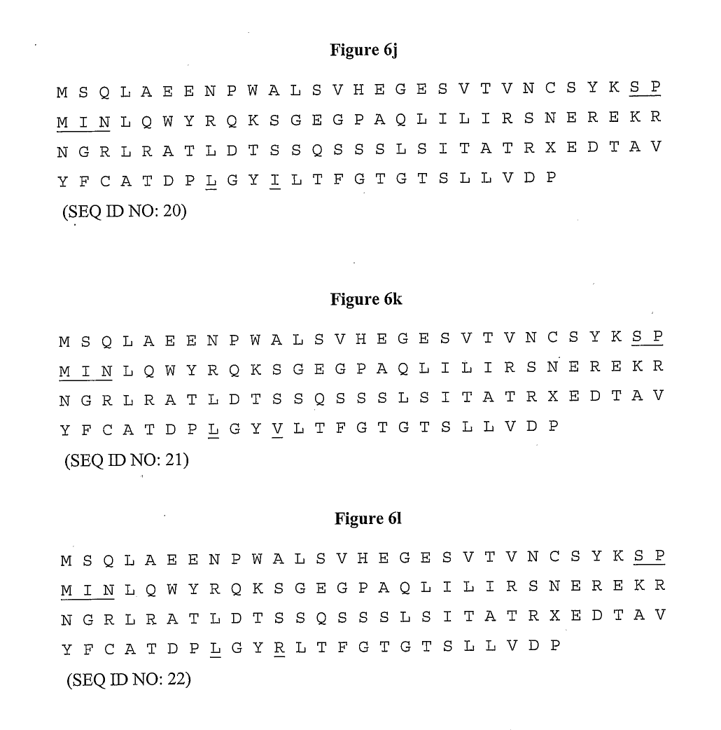

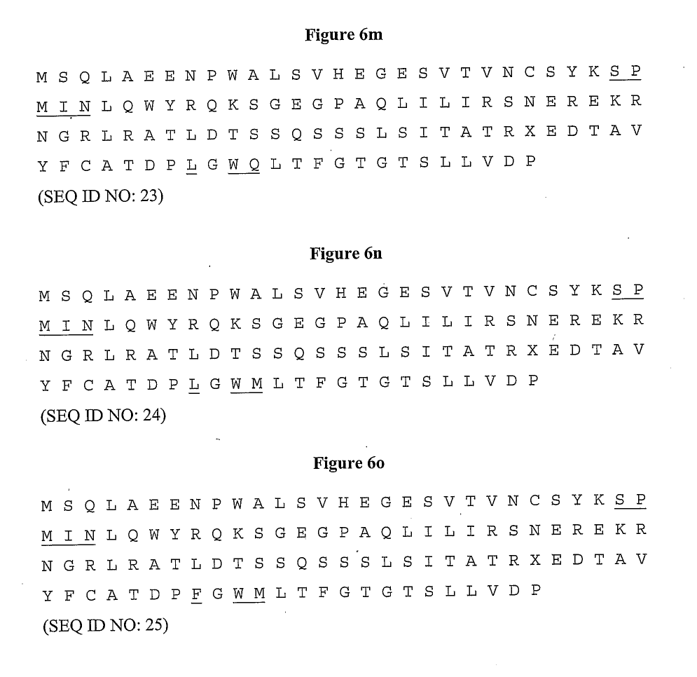

Further preferred embodiments of the invention are provided by TCRs comprising one of the mutated alpha chain variable region amino acid sequences shown in FIGS. 6a to 6o. (SEQ ID Nos: 11 to 25) Phenotypically silent variants of such TCRs also form part of this invention.

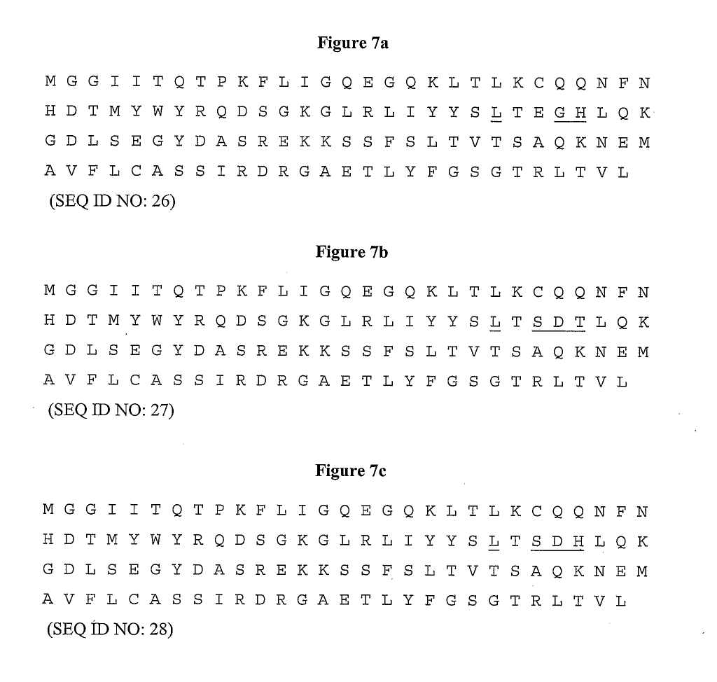

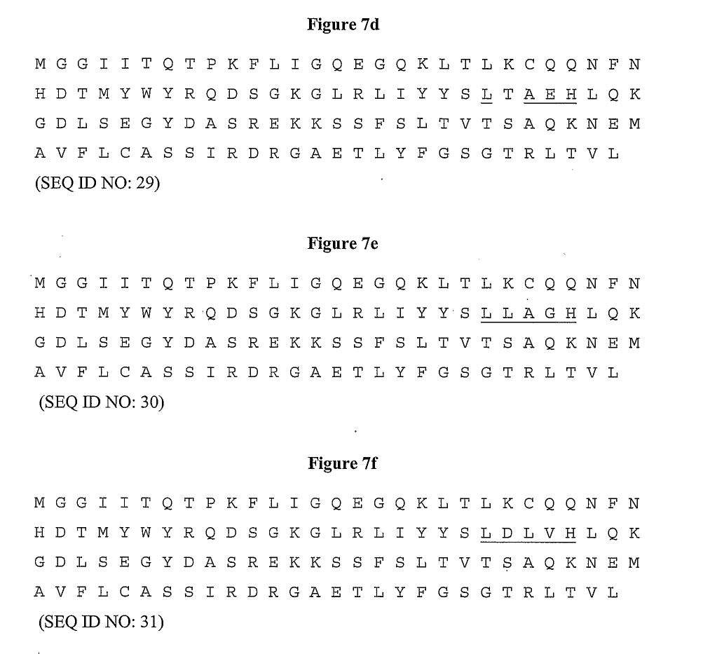

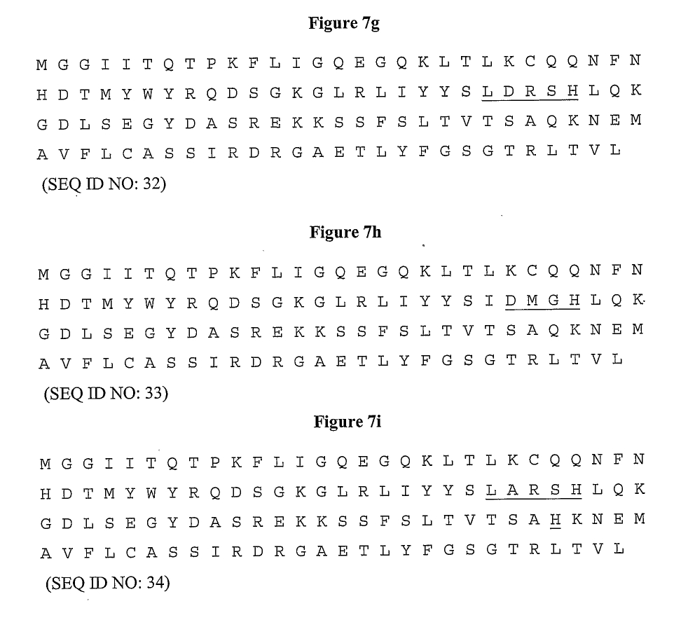

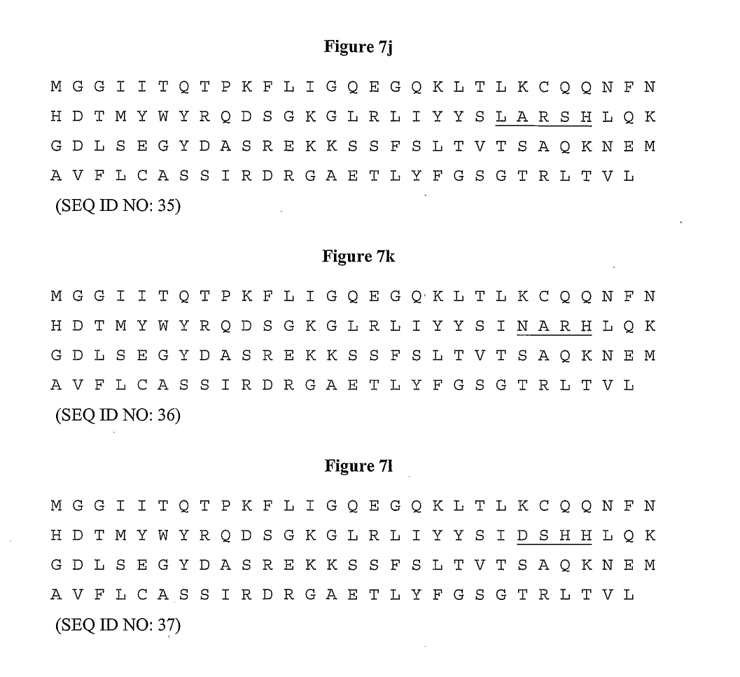

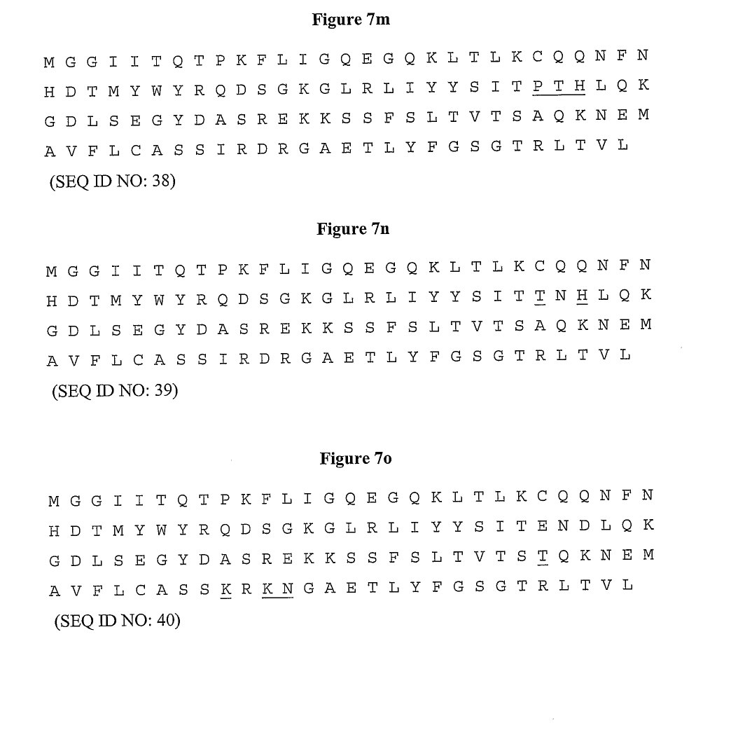

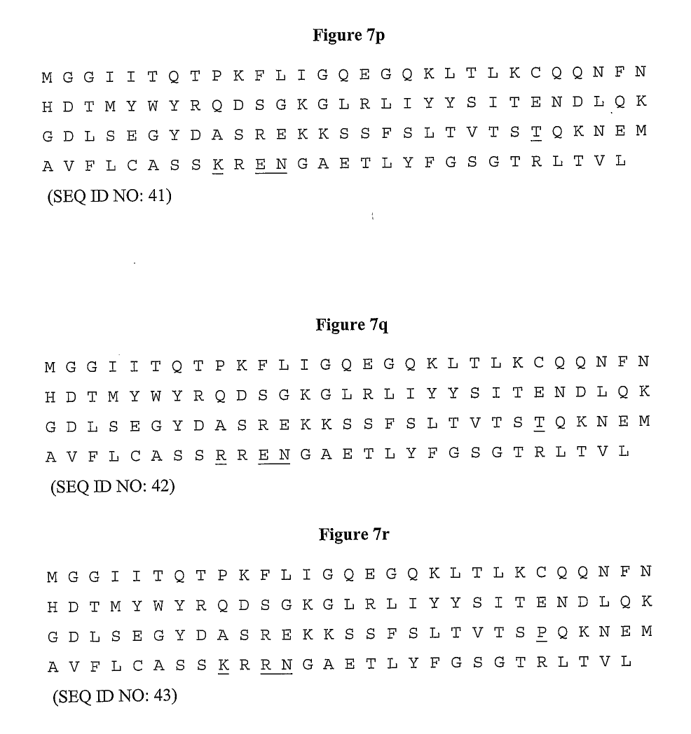

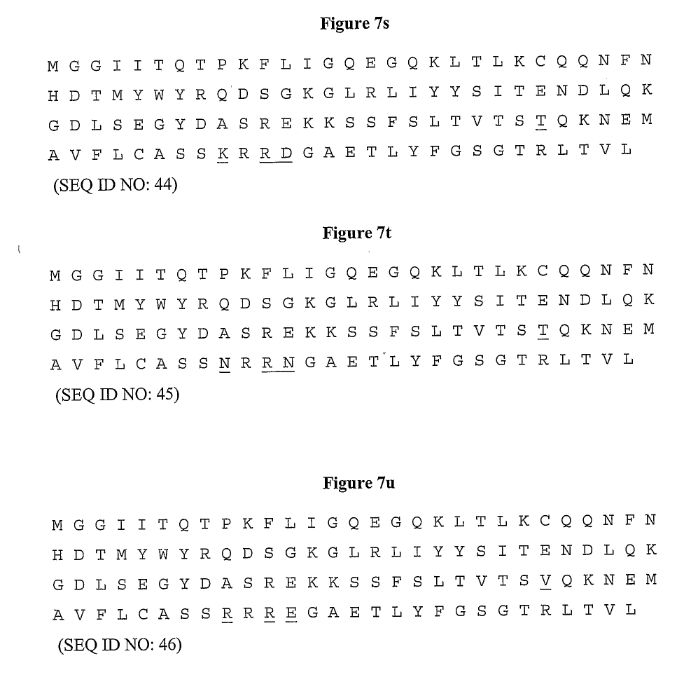

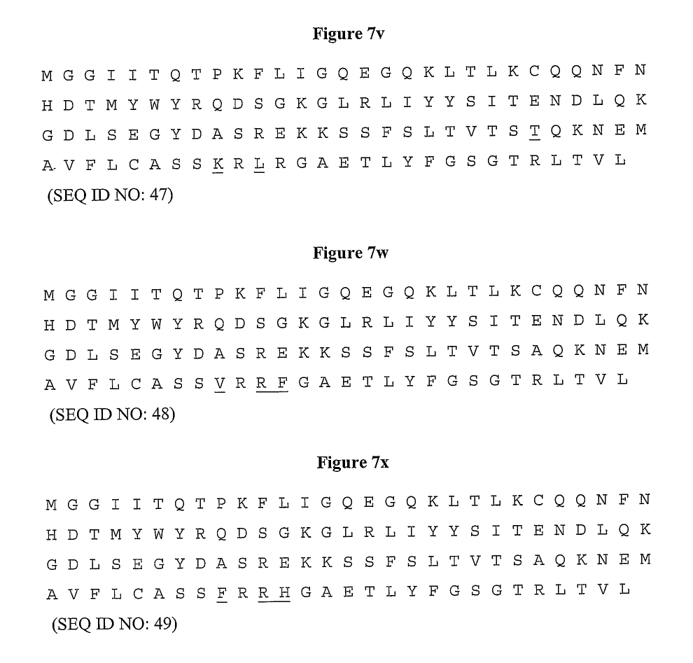

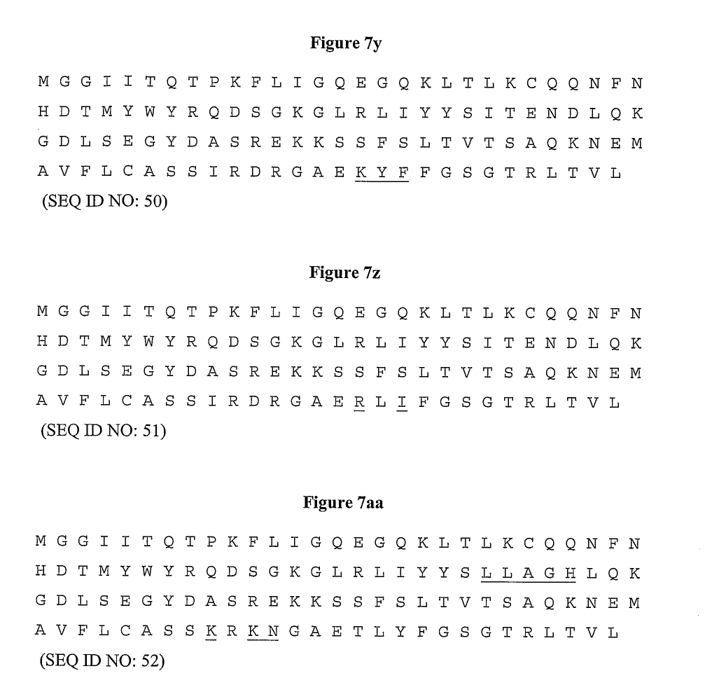

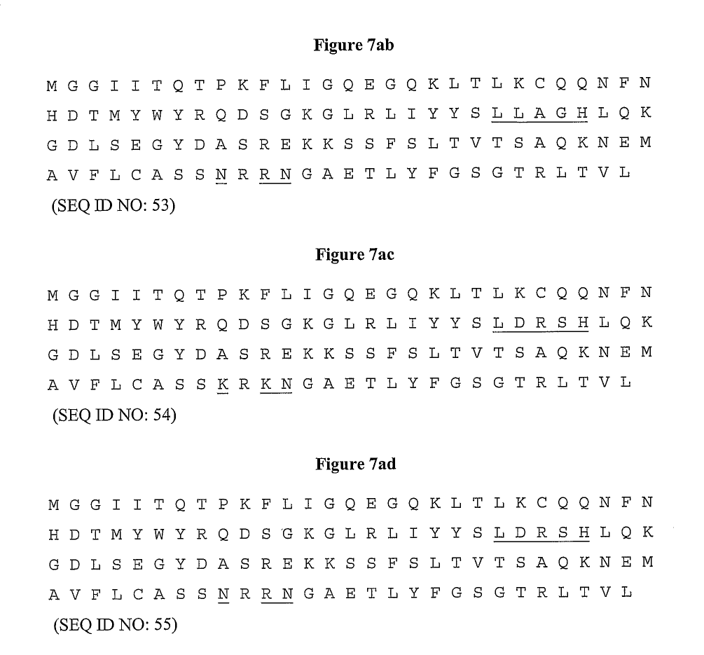

Additional preferred embodiments of the invention are provided by TCRs comprising one of the mutated beta chain variable region amino acid sequences shown in FIG. 7a to 7ad. (SEQ ID Nos: 26 to 55) Phenotypically silent variants of such TCRs also form part of this invention.

Further preferred embodiments are provided by TCRs of the invention comprising the alpha chain variable region amino acid sequence and the beta chain variable region amino acid sequence combinations listed below, phenotypically silent variants of such TCRs also form part of this invention:

| Alpha chain variable region | Beta chain variable region |
|---|---|
| sequence, | sequence, |
| SEQ ID NO: | SEQ ID NO: |
| 11 | 2 |
| 12 | 2 |
| 13 | 2 |
| 14 | 2 |
| 15 | 2 |
| 16 | 2 |
| 17 | 2 |
| 18 | 2 |
| 19 | 2 |
| 20 | 2 |
| 21 | 2 |
| 22 | 2 |
| 23 | 2 |
| 24 | 2 |
| 25 | 2 |
| 11 | 26 |
| 11 | 27 |
| 11 | 28 |
| 11 | 29 |
| 11 | 30 |
| 11 | 31 |
| 11 | 32 |
| 11 | 33 |
| 11 | 34 |
| 11 | 35 |
| 11 | 36 |
| 11 | 37 |
| 11 | 38 |
| 11 | 39 |
| 11 | 40 |
| 11 | 41 |
| 11 | 42 |
| 11 | 43 |
| 11 | 44 |
| 11 | 45 |
| 11 | 46 |
| 11 | 47 |
| 11 | 48 |
| 11 | 49 |
| 11 | 50 |
| 11 | 51 |
| 11 | 52 |
| 11 | 53 |
| 11 | 54 |
| 11 | 55 |
| 24 | 40 |
| 24 | 45 |
| 20 | 52 |

In a preferred embodiment TCRs of the invention comprise the alpha chain variable region amino acid sequence shown in SEQ ID NO: 20 and the beta chain variable region amino acid sequence shown in SEQ ID NO: 52.

In another preferred embodiment TCRs of the invention further comprise a TCR alpha chain constant domain sequence selected from the group consisting of:

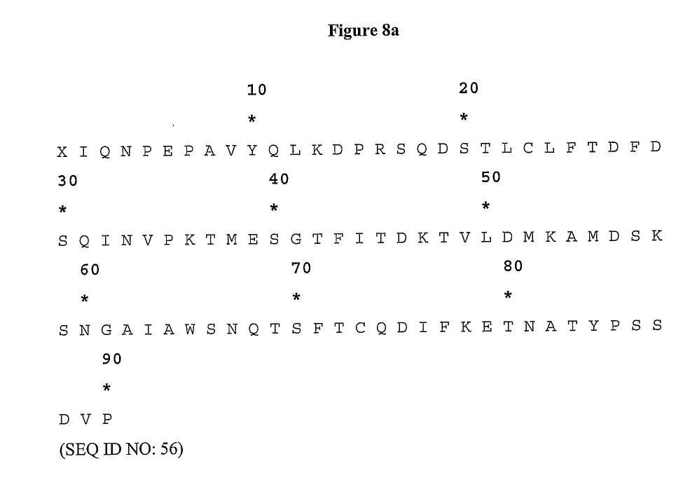

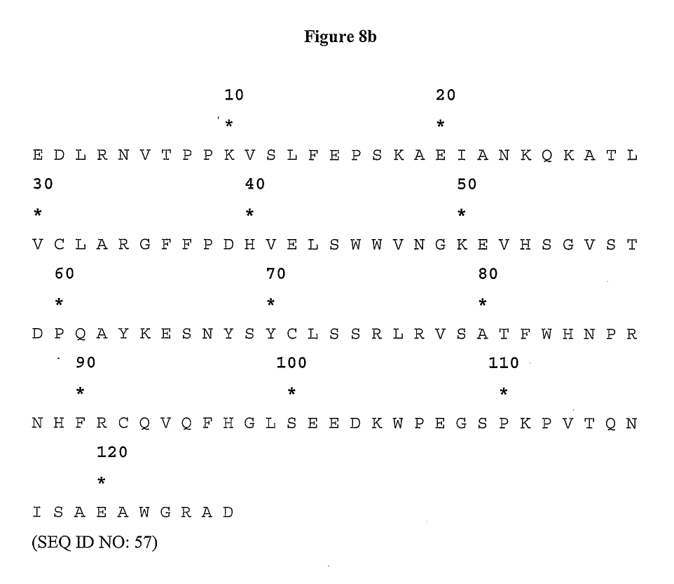

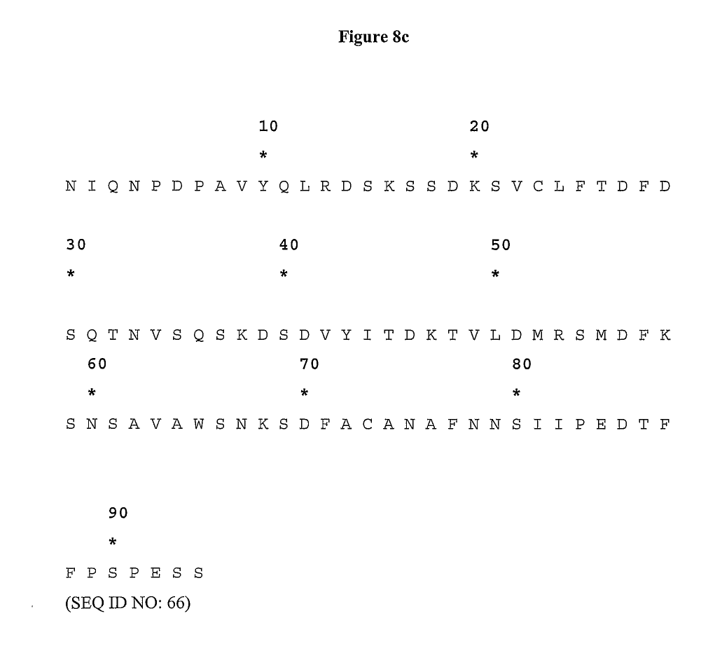

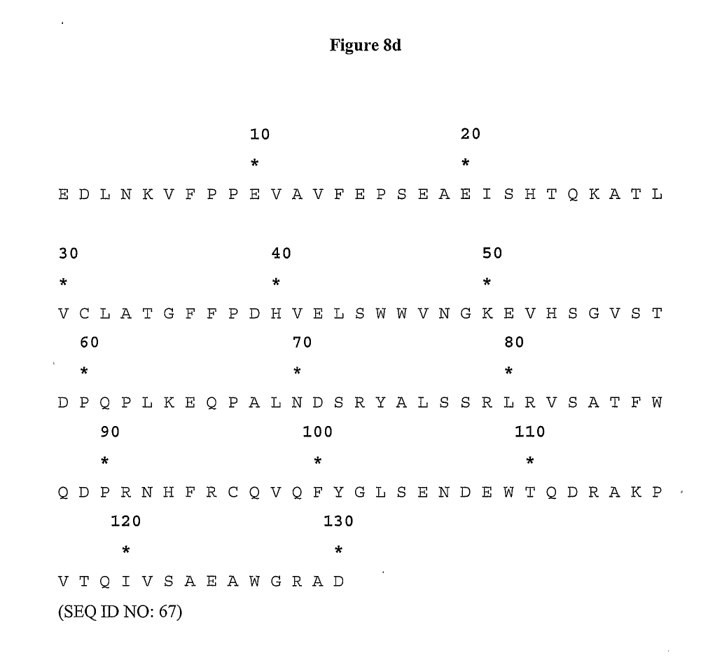

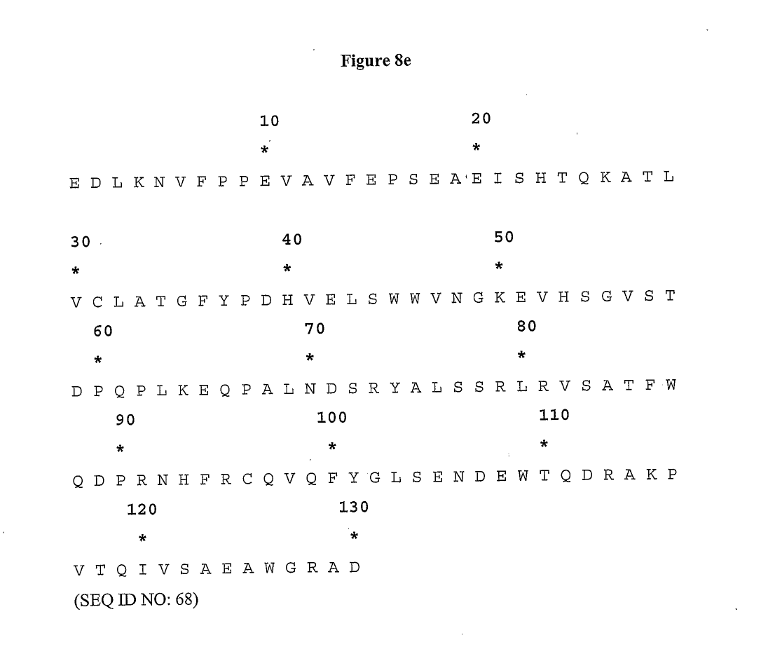

the murine alpha chain constant domain amino acid sequence shown in FIG. 8a (SEQ ID NO: 56), and
the human alpha chain constant domain amino acid sequence shown in FIG. 8c (SEQ ID NO: 66) or phenotypically silent variants of either of the foregoing sequences.

In a further preferred embodiment TCRs of the invention further comprise a TCR beta chain constant domain sequence selected from the group consisting of:

the murine beta chain amino acid constant domain sequence shown in FIG. 8b (SEQ ID NO: 57),
the human beta chain constant domain amino acid sequence shown in FIG. 8d (SEQ ID NO: 67), and
the human beta chain constant domain amino acid sequence shown in FIG. 8e (SEQ ID NO: 68) or phenotypically silent variants of any of the foregoing sequences.

As is known to those skilled in the art, part of the diversity of the TCR repertoire is due to variations which occur in the amino acid encoded by the codon at the boundary between the variable region, as defined herein, and the constant domain. For example, an Asparagine (N) amino acid residue is present at this boundary in the alpha chain of the wild-type murine insulin-specific TCR of the invention. In other wild-type murine TCR alpha chains this residue may be a Tyrosine (Y). This amino acid residue is represented by an “X” in the murine TCR alpha constant domain sequence shown in FIG. 8a (SEQ ID NO: 56).

As used herein the term “phenotypically silent variants” is understood to refer to those TCRs which have a K_{D} for the said LYLVCGERG-H-2K^{d} complex of less than or equal to 1 μM and/or have an off-rate (k_{off}) of 1×10⁻³ S⁻¹ or slower. For example, as is known to those skilled in the art, it may be possible to produce TCRs that incorporate minor changes in the constant domain and/or variable regions thereof compared to those detailed above without altering the affinity and/or off-rate for the interaction with the LYLVCGERG-H-2K^{d} complex. One specific example of such a change is provided by a variant of the TCR α alpha chain variable region sequence of SEQ ID NO: 14 in which residue 10 using the numbering of FIG. 1a (SEQ ID NO: 1) was mutated from the wild-type Tryptophan “W” to a Glutamine “Q” without altering the affinity or kinetics of soluble disulfide linked TCRs containing this mutation for the cognate the LYLVCGERG-H-2K^{d} complex. Such trivial variants are included in the scope of this invention. Those TCRs in which one or more conservative substitutions have been made also form part of this invention.

In one broad aspect, the TCRs of the invention are in the form of either single chain TCRs (scTCRs) or dimeric TCRs (dTCRs) as described in WO 04/033685 and WO 03/020763.

A suitable scTCR form comprises a first segment constituted by an amino acid sequence corresponding to a TCR α chain variable region sequence, a second segment constituted by an amino acid sequence corresponding to a TCR β chain variable region sequence fused to the N terminus of an amino acid sequence corresponding to a TCR β chain constant domain extracellular sequence, and a linker sequence linking the C terminus of the first segment to the N terminus of the second segment.

Alternatively the first segment may be constituted by an amino acid sequence corresponding to a TCR β chain variable region sequence, the second segment may be constituted by an amino acid sequence corresponding to a TCR α chain variable region sequence fused to the N terminus of an amino acid sequence corresponding to a TCR α chain constant domain extracellular sequence

The above scTCRs may further comprise a disulfide bond between the first and second chains, said disulfide bond being one which has no equivalent in native αβT cell receptors, and wherein the length of the linker sequence and the position of the disulfide bond being such that the variable domain sequences of the first and second segments are mutually orientated substantially as in native αβ T cell receptors.

More specifically the first segment may be constituted by an amino acid sequence corresponding to a TCR α chain variable region sequence fused to the N terminus of an amino acid sequence corresponding to a TCR α chain constant domain extracellular sequence, the second segment may be constituted by an amino acid sequence corresponding to a TCR β chain variable region fused to the N terminus of an amino acid sequence corresponding to TCR β chain constant domain extracellular sequence, and a disulfide bond may be provided between the first and second chains, said disulfide bond being one which has no equivalent in native αβ T cell receptors.

Certain embodiments are provided by scTCRs of the invention wherein the said first and second segments are linked by a disulfide bond between a pair of cysteine residues substituted for amino acid residues selected from the group consisting of:

Thr 48 of the murine alpha chain extracellular constant domain using the numbering of SEQ ID NO: 56 and Ser 57 of the murine TCR beta chain extracellular constant domain using the numbering of SEQ ID NO: 57, and
Thr 48 of the human TCR alpha chain extracellular constant domain using the numbering of SEQ ID NO: 66 and Ser 57 of the TCR beta chain extracellular constant domain using the numbering of SEQ ID NO: 67 or SEQ ID NO: 68.

In the above scTCR forms, the linker sequence may link the C terminus of the first segment to the N terminus of the second segment, and may have the formula -PGGG-(SGGGG)ₙ-P- wherein n is 5 or 6 and P is proline, G is glycine and S is serine:

(SEQ ID NO: 63)
-PGGG-SGGGGSGGGGSGGGGSGGGGSGGGG-P
(SEQ ID NO: 64)
-PGGG-SGGGGSGGGGSGGGGSGGGGSGGGGSGGGG-P

Suitable scTCR forms of the TCRs of the invention are provided wherein the TCR chain variable region sequences in the first and second segments correspond to the functional variable regions of a first TCR, and the TCR α or TCR β chain constant region extracellular sequence present in the second segment corresponds to that of a second TCR, the first and second TCRs being from different species. For example, the TCR chain variable region sequences in the first and second segments may correspond to the functional variable regions of a murine TCR, and the TCR α or TCR β chain constant region extracellular sequence present in the second segment corresponds to that of a human TCR.

A suitable dTCR form of the TCRs of the present invention comprises a first polypeptide wherein a sequence corresponding to a TCR α chain variable region sequence is fused to the N terminus of a sequence corresponding to a TCR α chain constant domain extracellular sequence, and a second polypeptide wherein a sequence corresponding to a TCR β chain variable region sequence is fused to the N terminus of a sequence corresponding to a TCR β chain constant domain extracellular sequence, the first and second polypeptides being linked by a disulfide bond which has no equivalent in native αβ T cell receptors, such as between a pair of cysteine residues substituted for amino acid residues selected from the group consisting of:

Thr 48 of the murine alpha chain extracellular constant domain using the numbering of SEQ ID NO: 56 and Ser 57 of the murine TCR beta chain extracellular constant domain using the numbering of SEQ ID NO: 57, and
Thr 48 of the human TCR alpha chain extracellular constant domain using the numbering of SEQ ID NO: 66 and Ser 57 of the TCR beta chain extracellular constant domain using the numbering of SEQ ID NO: 67 or SEQ ID NO: 68.

Suitable dTCR forms of the TCRs of the invention are provided wherein the TCR chain variable region sequences in the first and second polypeptides correspond to the functional variable domains of a first TCR, and the TCR chain constant region extracellular sequences present in the first and second polypeptide correspond to those of a second TCR, the first and second TCRs being from different species. For example, the TCR chain variable region sequences in the first and second polypeptides may correspond to the functional variable domains of a murine TCR, and the TCR chain constant region extracellular sequences present in the first and second polypeptide correspond to those of a human TCR.

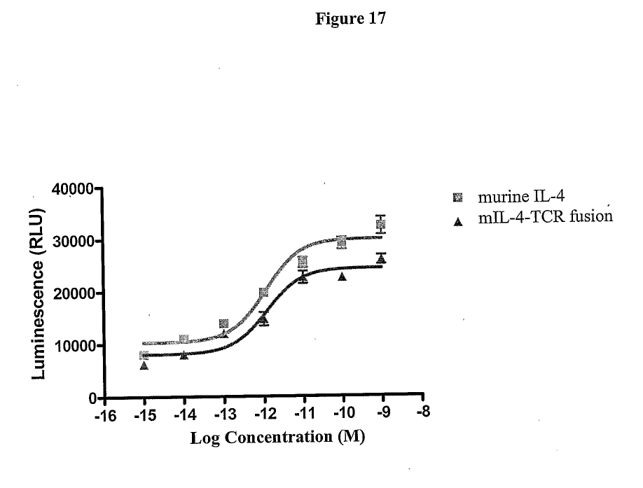

A specific embodiment of the invention is provided by a dTCR comprising the TCR chain sequence shown in FIG. 17a (SEQ ID NO: 60) and the TCR β chain sequence shown in FIG. 17b (SEQ ID NO: 61).

The above specified introduced disulfide interchain bonds having no equivalent in native TCRs in the dTCR or scTCR form of the TCRs are formed between cysteine residues corresponding to amino acid residues whose β carbon atoms are less than 0.6 nm apart in native TCRs. For example, as disclosed in WO03020763, between cysteine residues substituted for Thr 48 of exon 1 of human TRAC*01 and Ser 57 of exon 1 of human TRBC1*01 or TRBC2*01 or the murine equivalents thereof. Other sites where cysteines can be introduced to form such introduced disulfide bonds include the following:

| Human TCR | Human TCR | |
|---|---|---|
| Exon 1 | Exon 1 | Native β carbon |
| α chain | β chain | separation (nm) |
| Thr 45 | Ser 77 | 0.533 |
| Tyr 10 | Ser 17 | 0.359 |
| Thr 45 | Asp 59 | 0.560 |
| Ser 15 | Glu 15 | 0.59 |

The locations of the murine residues equivalent to the above human TCR constant domain residues are provided in the following tables, using the numbering of SEQ ID NO: 56 (FIG. 8a) for the murine TCR alpha chain and SEQ ID NO: 57 (FIG. 8b) for the murine TCR beta chain respectively:

| | Murine TCR α chain |
|---|---|
| Human TCR Exon 1 | residue of |
| α chain | SEQ ID NO: 56 |
| Thr 48 | Thr 48 |
| Thr 45 | Thr 45 |
| Tyr 10 | Tyr 10 |
| Ser 15 | Ser 17 |

| | Murine TCR β chain |
|---|---|
| Human TCR Exon 1 | residue of |
| β chain | SEQ ID NO: 57 |
| Ser 57 | Ser 57 |
| Ser 77 | Ser 74 |
| Ser 17 | Ser 17 |
| Asp 59 | Asp 59 |
| Glu 15 | Glu 15 |

The locations of the above human TCR constant domain residues are provided in the following tables, using the numbering of SEQ ID NO: 66 (FIG. 8c) for the human TCR alpha chain and SEQ ID NO: 67 (FIG. 8d) for the human TCR beta chain respectively:

| | Human TCR α chain |
|---|---|
| Human TCR Exon 1 | residue of |
| α chain | SEQ ID NO: 66 |
| Thr 48 | Thr 48 |
| Thr 45 | Thr 45 |
| Tyr 10 | Tyr 10 |
| Ser 15 | Ser 15 |

| | Human TCR β chain |
|---|---|
| Human TCR Exon 1 | residue of |
| β chain | SEQ ID NO: 67 |
| Ser 57 | Ser 57 |
| Ser 77 | Ser 77 |
| Ser 17 | Ser 17 |
| Asp 59 | Asp 59 |
| Glu 15 | Glu 15 |

The five specified locations for these introduced non-native interchain bonds are functionally equivalent, in that TCRs that include any one of these five interchain bonds can be produced in soluble forms. WO 03020763 describes the production of five soluble human HLA-A*0201 Tax-specific A6 TCRs each one of which contained a disulfide interchain bond selected from these five bond locations.

In certain embodiment the TCRs of the invention may include an interchain disulfide bond between residues corresponding to those linked by an interchain disulfide bond in native TCRs. As will be known to those skilled in the art the native interchain disulfide bond in human αβ TCRs is formed between a cysteine residue in the TCR α chain (amino acid 4, exon 2 of the TRAC*01 gene) and a cysteine residue in the TCR β chain (amino acid 2 of exon 2 for both the TRBC1*01 and TRBC2*01 genes)

Such a disulfide bond can be formed by adding single cysteine amino acid residues to the C termini of the murine TCR α chain constant domain sequence shown in SEQ ID NO: 56 and murine TCR β chain constant domain sequences provided by SEQ ID NO: 57, or by adding single cysteine amino acid residues to the C termini of the human TCR α chain constant domain sequence shown in SEQ ID NO: 66 and the human TCR β chain constant domain sequence provided by SEQ ID Nos: 67 or 68.

In certain embodiments the soluble dTCR or soluble scTCR of the invention does not contain a sequence corresponding to transmembrane or cytoplasmic sequences of native TCRs.

A preferred soluble TCR (sTCR) according to the invention comprises TCR α and TCR β chains truncated at the C-terminus thereof such that the cysteine residues which form the native interchain disulphide bond are excluded, i.e. truncated at the residue 1, 2, 3, 4, 5, 6, 7, 8, 9 or 10 residues N-terminal to the cysteine residues.

In one embodiment the sTCR of the invention comprises the whole of the TCR α chain which is N-terminal of exon 2, residue 4 of TRAC*01 and the whole of the TCR β chain which is N-terminal of exon 2, residue 2 of both TRBC1*01 and TRCB2*01.

In one embodiment a TCR of the invention may further comprise a reactive cysteine at the C-terminal or N-terminal of the TCR α chain or TCR β chain.

### PEGylated TCR Monomers

In one particular embodiment a TCR of the invention is associated with at least one polyalkylene glycol chain(s). This association may be cause in a number of ways known to those skilled in the art. In a preferred embodiment the polyalkylene chain(s) is/are covalently linked to the TCR. In a further embodiment the polyethylene glycol chains of the present aspect of the invention comprise at least two polyethylene repeating units.

### Multivalent TCR Complexes

One aspect of the invention provides a multivalent TCR complex comprising at least two TCRs of the invention. In one embodiment of this aspect, at least two TCR molecules are linked via linker moieties to form multivalent complexes. Preferably the complexes are water soluble, so the linker moiety should be selected accordingly. Furthermore, it is preferable that the linker moiety should be capable of attachment to defined positions on the TCR molecules, so that the structural diversity of the complexes formed is minimised. For example, said TCRs may be linked by a non-peptidic polymer chain or a peptidic linker sequence. One embodiment of the present aspect is provided by a TCR complex of the invention wherein the non-peptidic polymer chain or peptidic linker sequence extends between amino acid residues of each TCR which are not located in a variable region sequence of the TCR.

Since the complexes of the invention may be for use in medicine, the linker moieties should be chosen with due regard to their pharmaceutical suitability, for example their immunogenicity.

Examples of linker moieties which fulfil the above desirable criteria are known in the art, for example the art of linking antibody fragments.

There are two classes of linker that are preferred for use in the production of multivalent TCR molecules of the present invention. A TCR complex of the invention in which the TCRs are linked by a polyalkylene glycol chain provides one embodiment of the present aspect.

The most commonly used of this class are based on polyethylene glycol or PEG. WO 2004/050705 provides practical details concerning the construction of multivalent TCRs generally, using PEG linkers.

Peptidic linkers are the other class of TCR linkers. These linkers are comprised of chains of amino acids, and function to produce simple linkers or multimerisation domains onto which TCR molecules can be attached. The biotin/streptavidin system has previously been used to produce TCR tetramers (see WO 99/60119) for in-vitro binding studies. However, strepavidin is a microbially-derived polypeptide and as such not ideally suited to use in a therapeutic.

A TCR complex of the invention in which the TCRs are linked by a peptidic linker derived from a murine multimerisation domain provides a further embodiment of the present aspect.

A multivalent TCR complex of the invention comprising at least two TCRs provides a final embodiment of this aspect, wherein at least one of said TCRs is associated with a therapeutic agent.

In one aspect the TCR of the invention may be associated with a therapeutic agent or detectable moiety. For example, said therapeutic agent or detectable moiety may be covalently linked to the C- or N-terminus of one or both of the TCR chains.

In one embodiment of the invention said therapeutic agent or detectable moiety is covalently linked to the N-terminus of one or both TCR chains.

In one aspect the scTCR or one or both of the dTCR chains of TCRs of the present invention may be labelled with an detectable moiety, for example a label that is suitable for diagnostic purposes. Such labelled TCRs are useful in a method for detecting a LYLVCGERG-H-2K^{d} complex which method comprises contacting the TCR ligand with a TCR (or a multimeric high affinity TCR complex) specific for the TCR ligand; and detecting binding to the TCR ligand. In tetrameric TCR complexes formed for example, using biotinylated heterodimers, fluorescent streptavidin can be used to provide a detectable label. Such a fluorescently-labelled TCR tetramer is suitable for use in FACS analysis, for example to detect antigen presenting cells carrying the LYLVCGERG-H-2K^{d} complex for which these high affinity TCRs are specific. The detectable moiety may be any suitable moiety known to those skilled in the art. For example, said moiety may be an enzyme, a dye (such as a dye which provides an infra-red signal), an MRI-detectable reagent or a radionuclide.

Another manner in which the soluble TCRs of the present invention may be detected is by the use of TCR-specific antibodies, in particular monoclonal antibodies.

In a further aspect a TCR (or multivalent complex thereof) of the present invention may alternatively or additionally be associated with (e.g. covalently or otherwise linked to) a therapeutic agent which may be, for example, an immune effector molecule such as an interleukin or a cytokine. IL-4, IL-10 and IL-13 are example cytokines suitable for association with the TCRs of the present invention.

A multivalent TCR complex of the invention may have enhanced binding capability for a TCR ligand compared to a non-multimeric wild-type or T cell receptor heterodimer of the invention. Thus, the multivalent TCR complexes according to the invention are particularly useful for tracking or targeting cells presenting LYLVCGERG-H-2K^{d} complexes in vitro or in vivo, and are also useful as intermediates for the production of further multivalent TCR complexes having such uses. These TCRs or multivalent TCR complexes may therefore be provided in a pharmaceutically acceptable formulation for use in vivo.

The invention also provides a method for delivering a therapeutic agent to a target cell, which method comprises contacting potential target cells with a TCR or multivalent TCR complex in accordance with the invention under conditions to allow attachment of the TCR or multivalent TCR complex to the target cell, said TCR or multivalent TCR complex being specific for the LYLVCGERG-H-2K^{d} complex and having the therapeutic agent associated therewith.

In particular, the soluble TCR or multivalent TCR complex of the present invention can be used to deliver therapeutic agents to the location of cells presenting a particular antigen. This would be useful in many situations and, in particular, against the beta cells of subject suffering from T1DM or T2DM. A therapeutic agent could be delivered such that it would exercise its effect locally but not only on the cell to which it binds. Thus, one particular strategy envisages immunomodulatory molecules linked to TCRs or multivalent TCR complexes according to the invention specific for the LYLVCGERG-H-2K^{d} complex.

Many therapeutic agents could be employed for this use, for instance immune effector molecules (cytokines for example). To ensure that the therapeutic effects are exercised in the desired location the agents could be inside a liposome linked to streptavidin so that the compound is released slowly. This will ensure that the agent has maximum effect after binding of the TCR to the relevant antigen presenting cells.

One embodiment is provided by a membrane preparation comprising a TCR of the invention. Said membrane preparation may be prepared from cells or may comprise a synthetic membrane.

Another embodiment is provided by a cell harbouring an expression vector comprising nucleic acid encoding a TCR of the invention. For example, said cell may be a T cell.

Further embodiments of the invention are provided by a pharmaceutical composition comprising:

a TCR or a multivalent TCR complex of the invention (optionally associated with a therapeutic agent), together with a pharmaceutically acceptable carrier;
Therapeutic or imaging TCRs in accordance with the invention will usually be supplied as part of a sterile, pharmaceutical composition which will normally include a pharmaceutically acceptable carrier. This pharmaceutical composition may be in any suitable form, (depending upon the desired method of administering it to a patient). It may be provided in unit dosage form, will generally be provided in a sealed container and may be provided as part of a kit. Such a kit would normally (although not necessarily) include instructions for use. It may include a plurality of said unit dosage forms.

The pharmaceutical composition may be adapted for administration by any appropriate route, for example parenteral, transdermal or via inhalation, preferably a parenteral (including subcutaneous, intramuscular, or, most preferably intravenous) route. Such compositions may be prepared by any method known in the art of pharmacy, for example by mixing the active ingredient with the carrier(s) or excipient(s) under sterile conditions.

Dosages of the substances of the present invention can vary between wide limits, depending upon the disease or disorder to be treated, the age and condition of the individual to be treated, etc. and a physician will ultimately determine appropriate dosages to be used.

### Additional Aspects

A scTCR or dTCRs of the present invention may be provided in substantially pure form, or as a purified or isolated preparation. For example, it may be provided in a form which is substantially free of other proteins.

The sequence(s) of the nucleic acid or nucleic acids encoding the TCRs of the invention may be altered so as to optimise the level of expression obtained in the host cell. The host cell may be any appropriate prokaryotic or eukaryotic cell. For example, the host cell may be an E. coli cell or a murine T cell. The alterations made to these genetic sequences are silent that is they do not alter the amino acid sequence encoded. There are a number of companies which offer such expression optimisation services, including, GeneArt, Germany.

Preferred features of each aspect of the invention are as for each of the other aspects inutatis mutandis. The prior art documents mentioned herein are incorporated to the fullest extent permitted by law.

### EXAMPLES

The invention is further described in the following examples, which do not limit the scope of the invention in any way.

Reference is made in the following to the accompanying drawings in which:

FIGS. 1a and 1b are the alpha chain variable domain amino acid and beta chain variable domain amino acid sequences of the wild-type insulin-specific murine TCR respectively. The highlighted Cysteine (C) residue in the wild-type alpha chain sequence of FIG. 1a is routinely mutated to a Serine (S) to aid production from inclusion bodies of soluble versions of these TCRs. This Cysteine to Serine mutation is not associated with affinity maturation.

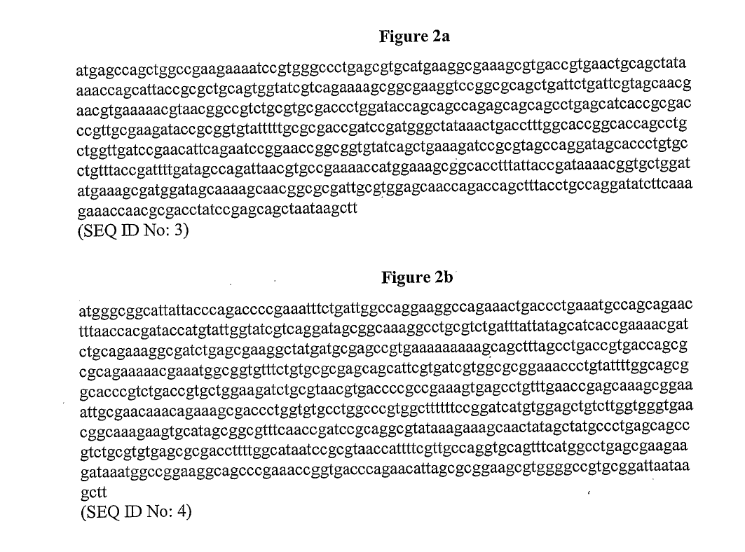

FIGS. 2a and 2b are DNA sequences encoding soluble versions of the wild-type insulin-specific murine TCR α and β chains respectively.

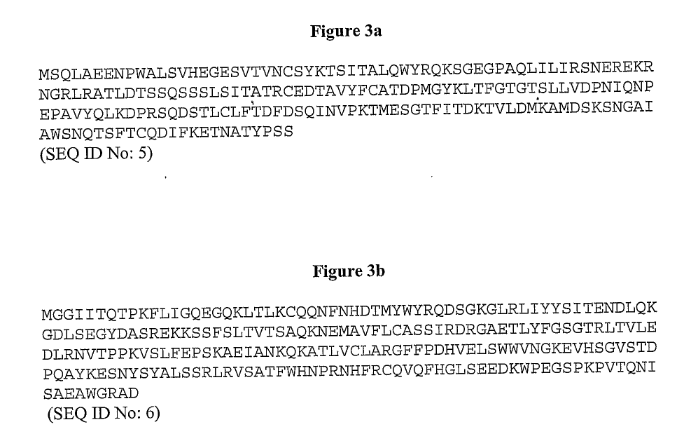

FIGS. 3a and 3b are the amino acid sequences of soluble versions of the wild-type insulin-specific murine TCR α and β chains produced from the DNA sequences of FIGS. 2a and 2b respectively.

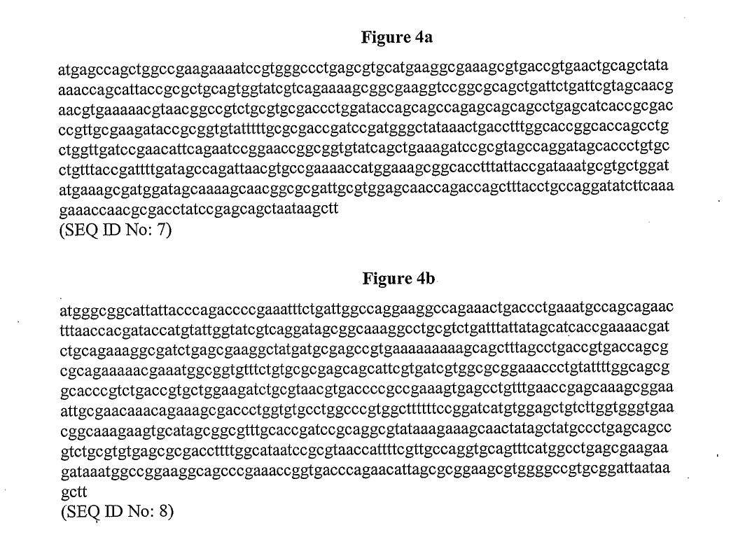

FIGS. 4a and 4b are DNA sequences encoding soluble versions of the wild-type insulin-specific murine TCR α and β chains mutated to encode additional cysteine residues to form a non-native disulfide bond respectively. The mutated codon is indicated by shading.

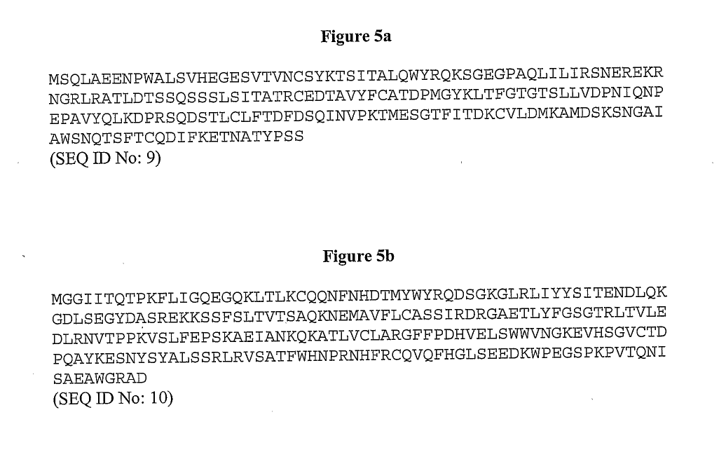

FIGS. 5a and 5b are amino acid sequences of the soluble insulin-specific murine TCR α and β chain sequences produced from the DNA sequences of FIGS. 4a and 4b. The introduced cysteine in each chain is indicated by shading.

FIG. 6 details the alpha chain variable region amino acid sequences of the high affinity insulin-specific murine TCR variants. The affinity-related mutations are underlined. The “X” residue may be a cysteine (C) as present in the wild-type insulin-specific murine TCR alpha chain, or a Serine (S) residue which has been shown to aid refolding.

FIG. 7 details the beta chain variable region amino acid sequences of the high affinity insulin-specific murine TCR variants. The mutated residues are underlined.

FIG. 8a details the amino acid sequence of a soluble portion of the murine TCR alpha chain constant domain. The amino acid residue “X” in this sequence may be an asparagine (N) or a tyrosine (Y).

FIG. 8b details the amino acid sequence of a soluble portion of the murine TCR beta chain constant domain.

FIG. 8c details the amino acid sequence of a soluble portion of the human TCR alpha chain constant domain.

FIG. 8d details the amino acid sequence of a soluble portion of a human TCR beta chain constant domain.

FIG. 8e details the amino acid sequence of a soluble portion of a second human TCR beta chain constant domain.

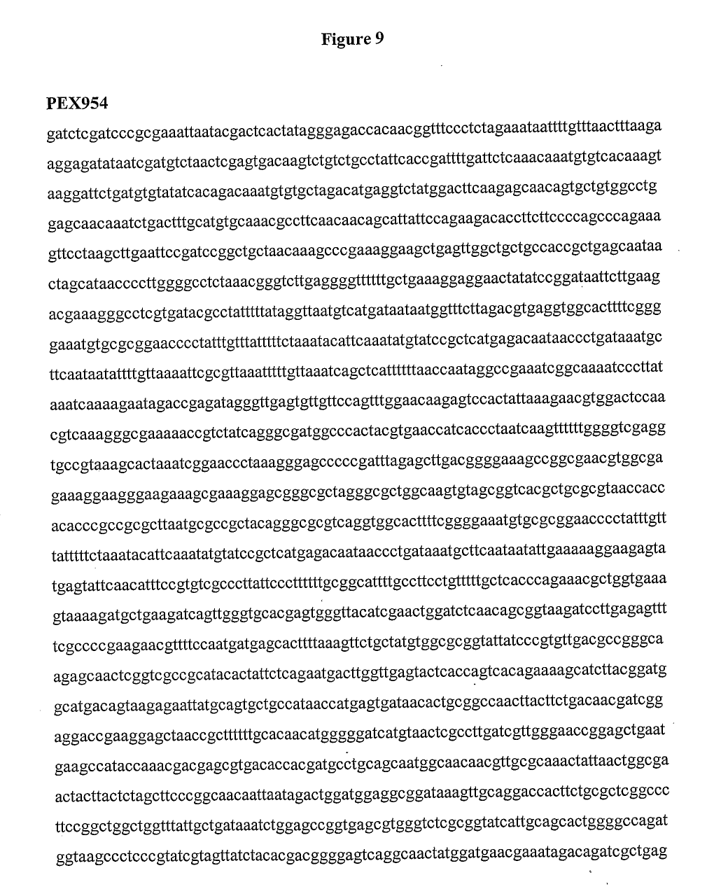

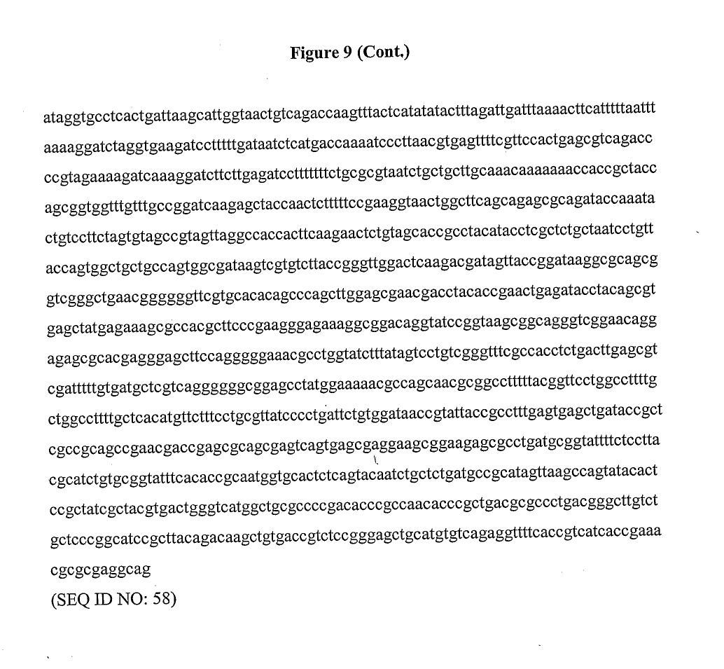

FIG. 9 details the DNA sequence of the pEX954 plasmid.

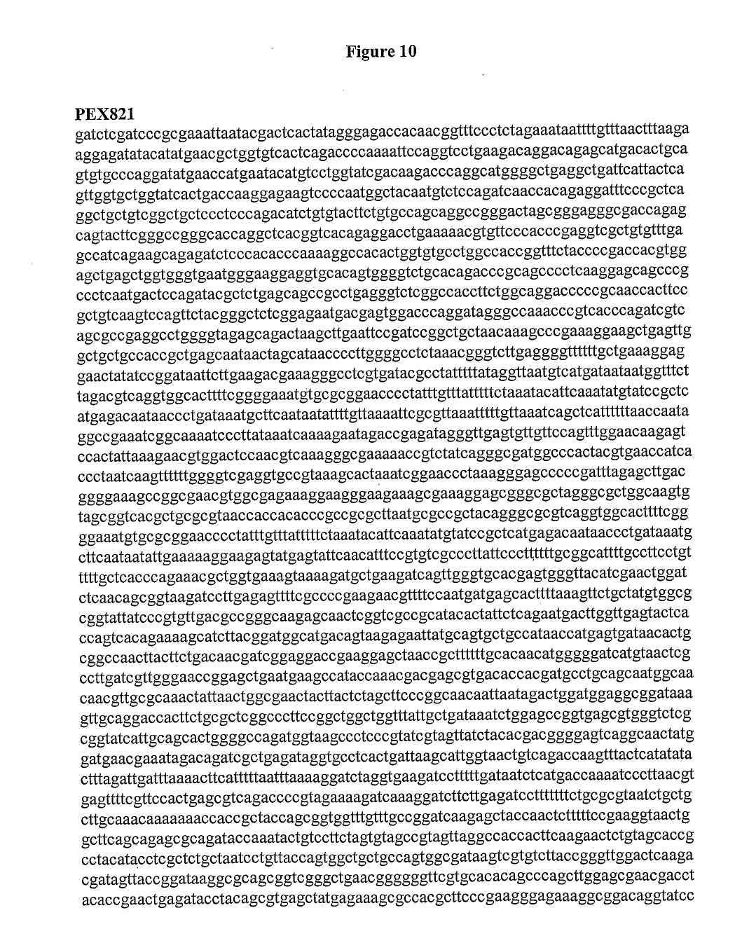

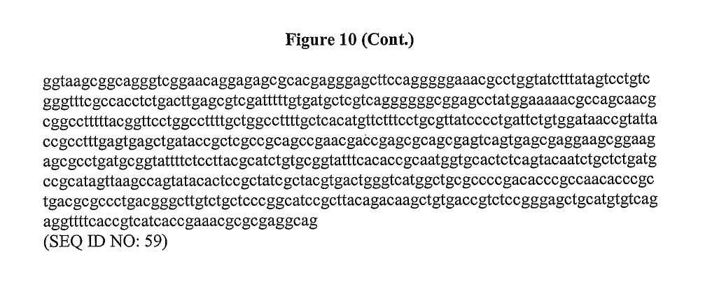

FIG. 10 details the DNA sequence of the pEX821 plasmid.

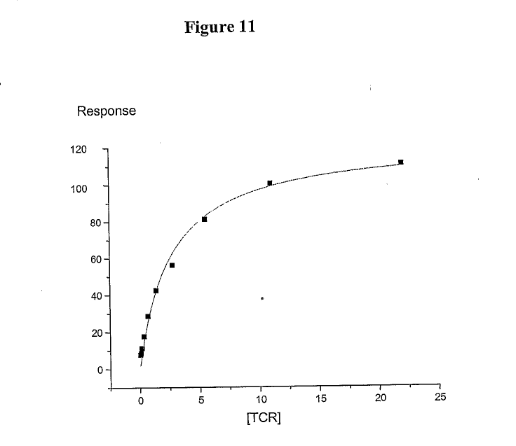

FIG. 11 is the Biacore response curve generated for the interaction of the soluble disulfide-linked wild-type insulin-specific murine TCR and the LYLVCGERL-H-2K^{d} complex.

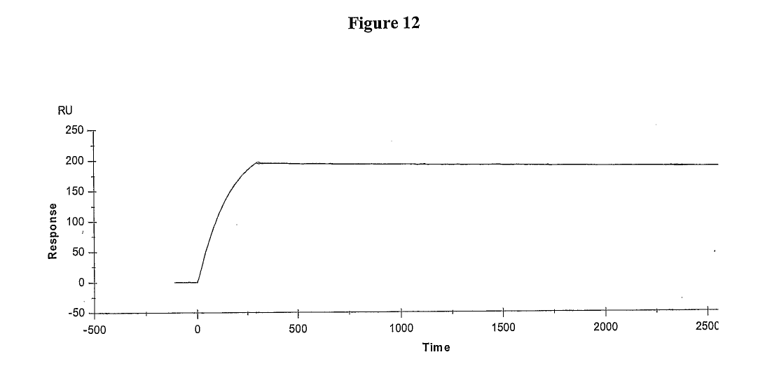

FIG. 12 is the Biacore response curve generated for the interaction of a soluble high affinity disulfide-linked insulin-specific murine TCR (alpha chain SEQ IDs Nos: 20 and 56 and beta chain SEQ Ms No: 52 and 57, but with introduced cysteines in the constant domains (substituted for Thr 48 of the murine alpha chain and Ser 57 of the murine beta chain, see page 18)—herein called “murine clone a12b46”) and the LYLVCGERL-H-2K^{d} complex.

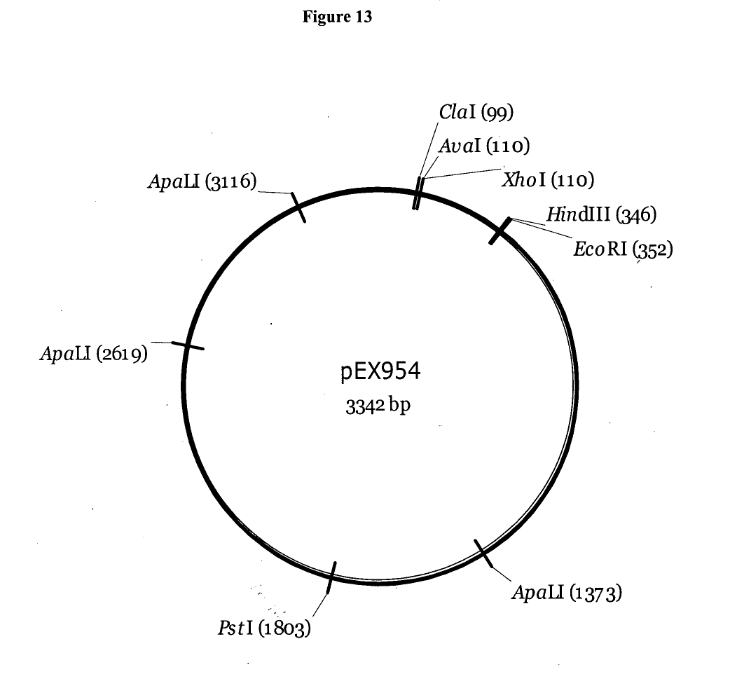

FIG. 13 provides a plasmid map of the pEX954 plasmid.

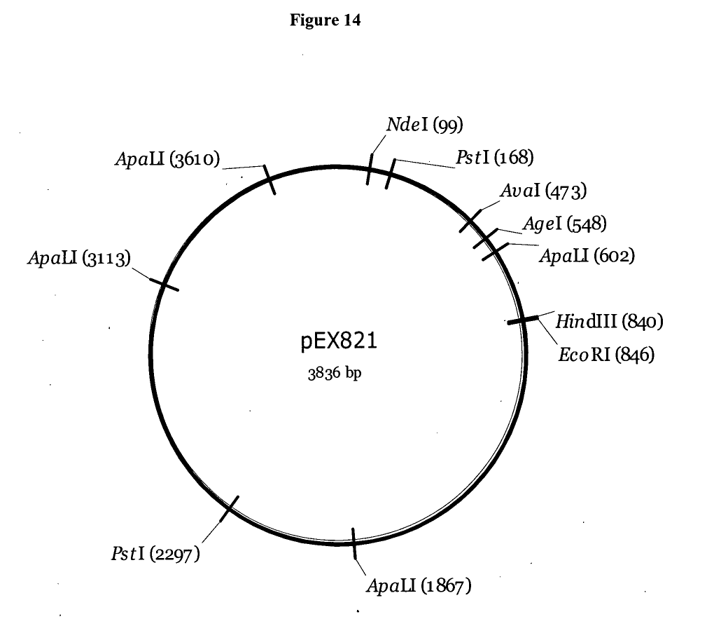

FIG. 14 provides a plasmid map of the pEX821 plasmid.

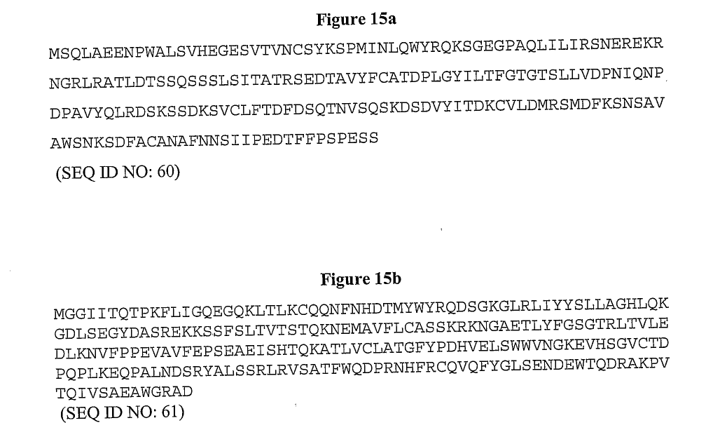

FIGS. 15a and 15b are the amino acids sequences of the alpha and beta chains of a soluble high affinity insulin-specific TCR having murine variable region sequences fused to human constant domain sequences (SEQ ID No: 60 and SEQ ID No: 61—herein called “chimeric clone a12b46”) The introduced cysteine residues are highlighted.

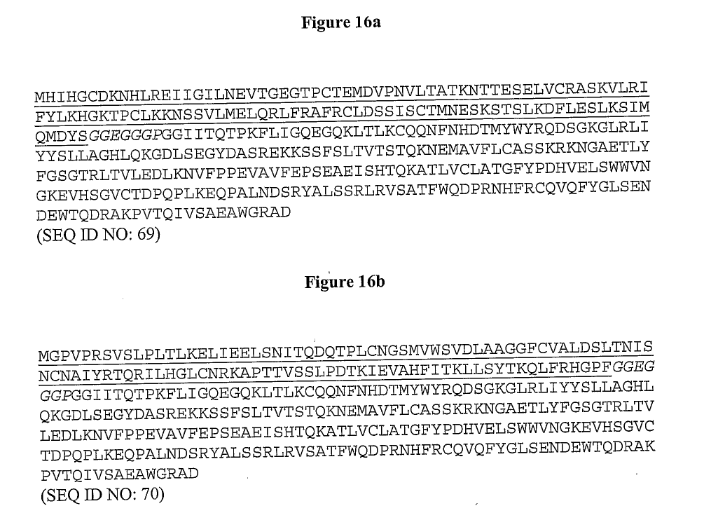

FIGS. 16a and 16b are the amino acids sequences of the beta chains of chimeric clone a12b46 fused to murine IL-4 and murine IL-13 respectively. The introduced cysteine residues in the TCR chains are highlighted, the linker sequences are in italics and the interleukin sequences are underlined.

FIG. 17 is a graph of the proliferative response of CTLL-2 cells to recombinant murine IL-4 (mIL-4) and a mIL-4-TCR fusion protein.

### Example 1

### Production of Soluble Disulfide-Linked TCRs Comprising the Variable Regions of a Wild-Type Insulin-Specific Murine TCR

FIGS. 4a and 4b are the DNA sequences encoding soluble disulfide-linked alpha beta chains of a wild-type insulin-specific murine TCR, which is specific for the LYLVCGERG-H-2K^{d} complex. These DNA sequences can be synthesis de-novo by a number of contract research companies, for example GeneArt (Germany).

Restriction enzyme recognition sites were added to these DNA sequences in order to facilitate ligation of these DNA sequences into the pGMT7-based expression plasmids, which contain the T7 promoter for high level expression in E. coli strain BL21-DE3(pLysS) (Pan et al., Biotechniques (2000) 29 (6): 1234-8)

The TCR alpha chain sequence was ligated into pEX954. (See FIGS. 9 and 13)

The TCR beta chain sequence was ligated into pEX821. (See FIGS. 10 and 14)

### Ligation

The cut TCR alpha and beta chain DNA and cut vector were ligated using a rapid DNA ligation kit (Roche) following the manufacturers instructions.

Ligated plasmids were transformed into competent E. coli strain XL1-blue cells and plated out on LB/agar plates containing 100 mg/ml ampicillin. Following incubation overnight at 37° C., single colonies were picked and grown in 10 ml LB containing 100 mg/ml ampicillin overnight at 37° C. with shaking. Cloned plasmids were purified using a Miniprep kit (Qiagen) and the insert was sequenced using an automated DNA sequencer (Lark Technologies).

FIGS. 5a and 5b are the soluble disulfide linked wild-type insulin-specific murine TCR α and β chain extracellular amino acid sequences produced from the DNA sequences of FIGS. 4a and 4b respectively.

### Example 2

### Production of High Affinity Variants of the Soluble Disulfide Linked Insulin-Specific Murine TCR

The soluble disulfide-linked wild-type insulin-specific murine TCR produced as described in Example 1 can be used a template from which to produce the TCRs of the invention which have an increased affinity for the LYLVCGERG-H-2K^{d} complex. Phage display is one means by which libraries of insulin-specific murine TCR variants can be generated in order to identify high affinity mutants. For example, the TCR phage display and screening methods described in (Li et al., (2005) Nature Biotech 23 (3): 349-354) can be adapted and applied to insulin-specific murine TCRs.

The amino sequences of the mutated TCR alpha and beta chain variable domains which, when combined with an appropriate TCR chain, demonstrate high affinity for the LYLVCGERG-H-2K^{d} complex, are listed in FIGS. 6 and 7 respectively. (SEQ ID Nos: 11-25 and 26-55 respectively) As is known to those skilled in the art the necessary codon changes required to produce these mutated chains can be introduced into the DNA encoding the corresponding wild-type insulin-specific murine TCR chains by site-directed mutagenesis. (QuickChange™ Site-Directed Mutagenesis Kit from Stratagene)

Briefly, this is achieved by using primers that incorporate the desired codon change(s) and the plasmids containing the relevant wild-type TCR chain DNA as a template for the mutagenesis:

Mutagenesis was carried out using the following conditions: 50 ng plasmid template, 1 μl of 10 mM dNTP, 5 μl of 10× Pfu DNA polymerase buffer as supplied by the manufacturer, 25 pmol of fwd primer, 25 pmol of rev primer, 1 μl pfu DNA polymerase in total volume 50 μl. After an initial denaturation step of 2 mins at 95 C, the reaction was subjected to 25 cycles of denaturation (95 C, 10 secs), annealing (55 C 10 secs), and elongation (72 C, 8 mins). The resulting product was digested with DpnI restriction enzyme to remove the template plasmid and transformed into E. coli strain XL1-blue. Mutagenesis was verified by sequencing.

### Example 3

### Expression, Refolding and Purification of Soluble TCR

The expression plasmids containing the insulin-specific murine TCR α-chain and β-chain respectively as prepared in Examples 1 or 2 were transformed separately into E. coli strain BL21pLysS, and single ampicillin-resistant colonies were grown at 37° C. in TYP (ampicillin 100 μg/ml) medium to OD₆₀₀ of 0.4 before inducing protein expression with 0.5 mM IPTG. Cells were harvested three hours post-induction by centrifugation for 30 minutes at 4000 rpm in a Beckman J-6B. Cell pellets were re-suspended in a buffer containing 50 mM Tris-HCl, 25% (w/v) sucrose, 1 mM NaEDTA, 0.1% (w/v) NaAzide, 10 mM DTT, pH 8.0. After an overnight freeze-thaw step, re-suspended cells were sonicated in 1 minute bursts for a total of around 10 minutes in a Milsonix XL2020 sonicator using a standard 12 mm diameter probe. Inclusion body pellets were recovered by centrifugation for 30 minutes at 13000 rpm in a Beckman J2-21 centrifuge. Three detergent washes were then carried out to remove cell debris and membrane components. Each time the inclusion body pellet was homogenised in a Triton buffer (50 mM Tris-HCl, 0.5% Triton-X100, 200 mM NaCI, 10 mM NaEDTA, 0.1% (w/v) NaAzide, 2 mM DTT, pH 8.0) before being pelleted by centrifugation for 15 minutes at 13000 rpm in a Beckman J2-21. Detergent and salt was then removed by a similar wash in the following buffer: 50 mM Tris-HCl, 1 mM NaEDTA, 0.1% (w/v) NaAzide, 2 mM DTT, pH 8.0. Finally, the inclusion bodies were divided into 30 mg aliquots and frozen at −70° C. Inclusion body protein yield was quantitated by solubilising with 6M guanidine-HCl and measurement with a Bradford dye-binding assay (PerBio).

Approximately 30 mg of TCR β chain and 30 mg of TCR α chain solubilised inclusion bodies were thawed from frozen stocks, samples were then mixed and the mixture diluted into 15 ml of a guanidine solution (6 M Guanidine-hydrochloride, 20 mM DTT (dithiothreitol), 10 mM Sodium Acetate, 10 mM EDTA), to ensure complete chain denaturation. The guanidine solution containing fully reduced and denatured TCR chains was then injected into 1 litre of the following refolding buffer: 100 mM Tris pH 8.5, 400 mM L-Arginine, 2 mM EDTA, the redox couple 5 mM reduced Glutathione, 0.5 mM oxidised Glutathione or the redox couple 2-mercaptoethylamine and cystamine (to final concentrations of 6.6 mM and 3.7 mM, respectively), 5M urea, 0.2 mM PMSF.

The redox couple is added approximately 5 minutes before addition of the denatured TCR chains. The solution was left for 5 hrs±15 minutes. The refolded TCR was dialysed in Spectrapor 1 membrane (Spectrum; Product No. 132670) against 10 L 10 mM Tris pH 8.1 at 5° C.±3° C. for 18-20 hours. After this time, the dialysis buffer was changed to fresh 10 mM Tris pH 8.1 (10 L) and dialysis was continued at 5° C.±3° C. for another 20-22 hours.

Soluble TCR (sTCR) was separated from degradation products and impurities by loading the dialysed refold onto a POROS 50HQ anion exchange column and eluting bound protein with a gradient of 0-500 mM NaCl over 50 column volumes using an Akta purifier (Pharmacia). Peak fractions were stored at 4° C. and analysed by Coomassie-stained SDS-PAGE before being pooled and concentrated. Finally, the sTCR was purified and characterised using a Superdex 200HR gel filtration column pre-equilibrated in PBS buffer. The'peak eluting at a relative molecular weight of approximately 50 kDa was pooled and concentrated prior to characterisation by BIAcore surface plasmon resonance analysis.

### Example 4

### Biacore Surface Plasmon Resonance Characterisation of Soluble Insulin-Specific Murine TCR Binding to the LYLVCGERL-H-2K^{d} Complex

A surface plasmon resonance biosensor (Biacore 3000™) was used to analyse the binding of soluble insulin-specific murine TCRs to the LYLVCGERL-H-2K^{d} complex. The LYLVCGERL (SEQ ID NO: 65) peptide, which is a variant of the naturally produced LYLVCGERG (SEQ ID NO: 62) murine insulin derived peptide, was used for peptide-pulsing as it forms a more stable complex with H-2K^{d} molecule, hence making pulsing cells with exogenous peptide more effective.

This was facilitated by producing single pMHC complexes (described below) which were immobilised to a streptavidin-coated binding surface in a semi-oriented fashion, allowing efficient testing of the binding of a soluble T-cell receptor to up to four different pMHC (immobilised on separate flow cells) simultaneously. Manual injection of pMHC complex allows the precise level of immobilised class 1 molecules to be manipulated easily.

Biotinylated class I H-2K^{d} complex molecules were refolded in vitro from bacterially-expressed inclusion bodies containing the constituent subunit proteins and synthetic peptide, followed by purification and in vitro enzymatic biotinylation (O'Callaghan et al. (1999) Anal. Biochem. 266: 9-15). H-2K^{d}-heavy chain was expressed with a C-terminal biotinylation tag which replaces the transmembrane and cytoplasmic domains of the protein in an appropriate construct. Inclusion body expression levels of ˜75 mg/litre bacterial culture were obtained. The human MHC light-chain or β2-microglobulin was also expressed as inclusion bodies in E. coli from an appropriate construct, at a level of ˜500 mg/litre bacterial culture.

E. coli cells were lysed and inclusion bodies are purified to approximately 80% purity. Protein from inclusion bodies was denatured in 6 M guanidine-HCl, 50 mM Tris pH 8.1, 100 mM NaCl, 10 mM DTT, 10 mM EDTA, and was refolded at a concentration of 30 mg/litre heavy chain, 30 mg/litre β2m into 0.4 M L-Arginine-HCl, 100 mM Tris pH 8.1, 3.7 mM cystamine, 6.6 mM β-cysteamine, 4 mg/L of the LYLVCGERL (SEQ ID NO: 65) peptide required to be loaded by the H-2K^{d} molecule, by addition of a single pulse of denatured protein into refold buffer at <5° C. Refolding was allowed to reach completion at 4° C. for at least 1 hour.

Buffer was exchanged by dialysis in 10 volumes of 10 mM Tris pH 8.1. Two changes of buffer were necessary to reduce the ionic strength of the solution sufficiently. The protein solution was then filtered through a 1.5 μm cellulose acetate filter and loaded onto a POROS 50HQ anion exchange column (8 ml bed volume). Protein was eluted with a linear 0-500 mM NaCl gradient LYLVCGERL-H-2K^{d} complex eluted at approximately 250 mM NaCl, and peak fractions were collected, a cocktail of protease inhibitors (Calbiochem) was added and the fractions were chilled on ice.

Biotinylation tagged pMHC molecules were buffer exchanged into 10 mM Tris pH 8.1, 5 mM NaCl using a Pharmacia fast desalting column equilibrated in the same buffer. Immediately upon elution, the protein-containing fractions were chilled on ice and protease inhibitor cocktail (Calbiochem) was added. Biotinylation reagents were then added: 1 in M biotin, 5 mM ATP (buffered to pH 8), 7.5 mM MgCl2, and 5 μg/ml BirA enzyme (purified according to O'Callaghan et al. (1999) Anal. Biochem. 266: 9-15). The mixture was then allowed to incubate at room temperature overnight.

The biotinylated LYLVCGERL-H-2K^{d} molecules were purified using gel filtration chromatography. A Pharmacia Superdex 75 HR 10/30 column was pre-equilibrated with filtered PBS and 1 ml of the biotinylation reaction mixture was loaded and the column was developed with PBS at 0.5 ml/min. Biotinylated LYLVCGERL-H-2K^{d} molecules eluted as a single peak at approximately 15 ml. Fractions containing protein were pooled, chilled on ice, and protease inhibitor cocktail was added. Protein concentration was determined using a Coomassie-binding assay (PerBio) and aliquots of biotinylated LYLVCGERL-H-2K^{d} molecules were stored frozen at −20° C. Streptavidin was immobilised by standard amine coupling methods.

Such immobilised complexes are capable of binding T-cell receptors, which may be injected in the soluble phase The pMHC binding properties of sTCR are observed to be qualitatively and quantitatively similar if sTCR is used either in the soluble or immobilised phase. This is an important control for partial activity of soluble species and also suggests that biotinylated pMHC complexes are biologically as active as non-biotinylated complexes.

The interactions between soluble insulin-specific murine TCRs containing a novel inter-chain bond and a variant (LYLVCGERL-H-2K^{d}) of their cognate pMHC ligand complex ligand or an irrelevant MHC-peptide combination (an IGRP-derived peptide presented by H-2K^{d}, which sequence is VYLKTNVFL (SEQ ID No: 71)), the production of which is described above, were analysed on a Biacore 3000™ surface plasmon resonance (SPR) biosensor. SPR measures changes in refractive index expressed in response units (RU) near a sensor surface within a small flow cell, a principle that can be used to detect receptor ligand interactions and to analyse their affinity and kinetic parameters. The BIAcore experiments were performed at a temperature of 25° C., using PBS buffer (Sigma, pH 7.1-7.5) as the running buffer and in preparing dilutions of protein samples. Streptavidin was immobilised to the flow cells by standard amine coupling methods. The pHLA complexes were immobilised via the biotin tag. The assay was then performed by passing sTCR over the surfaces of the different flow cells at a constant flow rate, measuring the SPR response in doing so.

### To Measure Equilibrium Binding Constant

Serial dilutions of the wild-type or mutated insulin-specific murine TCRs were prepared and injected at constant flow rate of 5 μl min-1 over two different flow cells; one coated with ˜1000 RU of specific LYLVCGERL-H-2K^{d} complex, the second coated with ˜1000 RU of a non-specific IGRP-derived peptide (which sequence is VYLKTNVFL (SEQ ID No: 71)-H-2K^{d} complex. Response was normalised for each concentration using the measurement from the control cell. Normalised data response was plotted versus concentration of TCR sample and fitted to a hyperbola in order to calculate the equilibrium binding constant, K_{D}. (Price & Dwek, Principles and Problems in Physical Chemistry for Biochemists (2^{nd} Edition) 1979, Clarendon Press, Oxford).

### To Measure Kinetic Parameters

For high affinity TCRs K_{D} was determined by experimentally measuring the dissociation rate constant, kd, and the association rate constant, ka. The equilibrium constant K_{D} was calculated as kd/ka.

TCR was injected over two different cells one coated with ˜300 RU of specific LYLVCGERL-H-2K^{d} complex, the second coated with ˜300 RU of non-specific IGRP-derived peptide (VYLKTNVFL (SEQ ID No: 71)-H-2K^{d} complex. Flow rate was set at 50 μl/min. Typically 250 μl of TCR at ˜3 μM concentration was injected. Buffer was then flowed over until the response had returned to baseline. Kinetic parameters were calculated using Biaevaluation software. The dissociation phase was also fitted to a single exponential decay equation enabling calculation of half-life.

### Results

The interaction between a soluble disulfide-linked wild-type insulin-specific murine TCR (consisting of the TCR α and β chains detailed in SEQ ID NOs: 9 and 10 respectively) and the LYLVCGERL-H-2K^{d} complex was analysed using the above methods and demonstrated a K_{D} of 2.6 μM and an off-rate (k_{off}) of approximately 0.69 S⁻¹ or slower. (See FIG. 11 for Biacore response curve)

The TCRs having murine α and β chains and the variable domains specified in the following table have a K_{D} of less than or equal to 1 μM and/or a k_{off} of 0.5 S⁻¹ or slower for the LYLVCGERL-H-2K^{d} complex.

| Alpha chain variable region | Beta chain variable region |
|---|---|
| SEQ ID NO: | SEQ ID NO: |
| 11 | 2 |
| 12 | 2 |
| 13 | 2 |
| 14 | 2 |
| 15 | 2 |
| 16 | 2 |
| 17 | 2 |
| 18 | 2 |
| 19 | 2 |
| 20 | 2 |
| 21 | 2 |
| 22 | 2 |
| 23 | 2 |
| 24 | 2 |
| 25 | 2 |
| 11 | 26 |
| 11 | 27 |
| 11 | 28 |
| 11 | 29 |
| 11 | 30 |
| 11 | 31 |
| 11 | 32 |
| 11 | 33 |
| 11 | 34 |
| 11 | 35 |
| 11 | 36 |
| 11 | 37 |
| 11 | 38 |
| 11 | 39 |
| 11 | 40 |
| 11 | 41 |
| 11 | 42 |
| 11 | 43 |
| 11 | 44 |
| 11 | 45 |
| 11 | 46 |
| 11 | 47 |
| 11 | 48 |
| 11 | 49 |
| 11 | 50 |
| 11 | 51 |
| 11 | 52 |
| 11 | 53 |
| 11 | 54 |
| 11 | 55 |
| 24 | 40 |
| 24 | 45 |
| 20 | 52 |

The murine clone a12b46 (alpha chain SEQ IDs Nos:SEQ 20 56 and beta chain SEQ IDs Nos: 52 and 57) had a K_{D} of 26 pM and/or a k_{off} of 1.6×10E-5 S⁻¹ or slower for the LYLVCGERL-H-2K^{d} complex.

Analogues of the murine TCRs listed in the above Table, and of the murine clone a12b46, which analogues have human rather than murine constant domains, also have the high affinity and/or slow off-rates which characterize the TCRs of the invention.

As noted above the foregoing study was carried out using LYLVCGERL peptide. That peptide differs from the native LYLVCGERG peptide in that the C-terminal amino acid is Leucine (L) instead of Glycine (G). The high affinity murine insulin-specific TCRs which successfully bound the variant peptide-MHC complex (LYLVCGERL-H-2K^{d}) will bind the wild-type peptide-MHC complex (LYLVCGERG-H-2K^{d}) since the altered C-terminal amino acid is a trivial variation.

### Example 5

### Quantification of Cell Surface TCR Ligands by Fluorescence Microscopy Using a High Affinitychimeric Clone a12b46 Murine Insulin-Specific TCR

The number of LYLVCGERL-H-2K^{d} antigens on peptide-pulsed NIT-1 murine beta cell insulinnoma cells and peptide-pulsed MH-S murine alVeolar macrophage cells was determined (on the assumption that one fluorescence signal relates to a single labeled TCR bound to its cognate pMHC ligand on the surface of the target cell) by single molecule fluorescence microscopy using a soluble biotinylated high affinity chimeric clone a12b46 insulin-specific TCR. This was facilitated by using biotinylated TCR to target the NIT-1 or MH-S cells and subsequent labeling of cell-bound TCR by streptavidin-R phycoerythrin (PE) conjugates. Individual PE molecules were then imaged by 3-dimensional fluorescence microscopy.

The LYLVCGERL (SEQ ID NO: 65) peptide, which is a variant of the naturally produced LYLVCGERG murine insulin derived peptide, was used for peptide-pulsing as it forms a more stable complex with H-2K^{d} molecule, hence making pulsing cells with exogenous peptide more effective.

FIGS. 15a and 15b (SEQ ID NOs: 60 and 61) respectively provide the amino acids sequences of alpha and beta chains of the soluble high affinity chimeric clone a12b46 chimeric insulin-specific TCR used. The variable regions of this TCR are derived from a murine TCR and the constant regions of this TCR are derived from a human TCR.

Staining of suspension cells. The NIT-1 or MH-S cells were incubated with peptide (10⁻⁵M) in culture medium Ham's F12K media (Fisher, UK) supplemented with 10% dialysed foetal bovine serum (FBS) for 90 minutes at 37° C., 5% CO₂. Cells were centrifuged (250×g for 10 minutes), media was removed, and cells washed twice with 500 μl of PBS supplemented with 400 μM MgCl₂, 400 μM CaCl₂ and 0.5% BSA (PBS/Mg/Ca). Cells were incubated in 200 μl of TCR solution (5 μg ml⁻¹ soluble biotinylated soluble high affinity chimeric clone a12b46 insulin-specific TCR, or 5 μg ml⁻¹ of an “irrelevant” biotinylated HLA-A*0201-NY-ESO peptide-specific high affinity TCR, in PBS/Mg/Ca containing 0.5% BSA albumin) for 30 min at 4° C. Additionally, an excess of unbiotinylated high affinity chimeric clone a12b46 insulin-specific TCR was added to control samples in order to specifically block the binding of the biotinylated soluble high affinity chimeric clone a12b46 insulin-specific TCR to the target LYLVCGERL-H-2K^{d} antigens. TCR solution was removed, and cells were washed three times with 500 μl of PBS/Mg/Ca. Cells were incubated in 200 μl of streptavidin-PE solution (5 μml⁻¹ streptavidin-PE in PBS/Mg/Ca containing 0.5% BSA) at room temperature in the dark for 20 min. Streptavidin-PE solution was removed and cells were washed five times with 500 μl of PBS/Mg/Ca. Cells were re-suspended in 400 μl of imaging media (R10) and transferred to chambered cover-slides before imaging by fluorescence microscopy.

Fluorescence microscopy. Fluorescent microscopy was carried out using an Axiovert 200M (Zeiss) microscope with a 63× Oil objective (Zeiss). A Lambda LS light source containing a 300W Xenon Arc lamp (Sutter) was used for illumination, and light intensity was reduced to optimal levels by placing a 0.3 and a 0.6 neutral density filter into the light path. Excitation and emission spectra were separated using a TRITC/DiI filter set (Chroma). Cells were imaged in three dimensions by z-stack acquisition (21 planes, 0.7 μm apart). Image acquisition and analysis was performed using Metamorph software (Universal Imaging) as described (Irvine et al., Nature (419), p 845-9, and Purbhoo et al., Nature Immunology (5), p 524-30.).

### Results

The peptide-pulsed NIT-1 and MH-S cells were specifically labelled by the biotinylated soluble high affinity chimeric clone a12b46 insulin-specific TCR, but not by the irrelevant biotinylated HLA-A*0201-NY-ESO peptide-specific TCR.

### Example 6

### Cellular Assay to Determine the Level of Biological Activity of the Murine IL-4 Part of a High Affinity Chimeric Clone a12b46 Murine Insulin-Specific TCR-Murine IL-4 Fusion Protein

The level of biological activity of the murine IL-4 part of a soluble high affinity chimeric clone a12b46 insulin-specific TCR-murine IL-4 fusion protein (“mIL-4-TCR fusion”) was determined by comparing the proliferative responses of IL-4R⁺ cells to the mIL-4-TCR fusion against those of the same cells to recombinant murine IL-4. The proliferative responses of these cells to the mIL-4-TCR fusion and recombinant murine IL-4 reagents were measured using a luciferase-based luminescence assay which quantified the total amount of ATP present in the cell cultures being tested. The assumption being that the total amount of ATP in the cell culture is proportional to the number of viable cells present.

The TCR part of the mIL-4-TCR fusion contains the alpha and beta chain variable region sequences of a murine TCR fused to alpha and beta chain constant domain sequences of a human TCR. The amino acid sequences of the TCR alpha chain and the TCR beta chain fused to murine IL-4 of the mIL-4-TCR fusion are given by FIGS. 15a (SEQ ID NO: 60) and 16a (SEQ ID NO: 69) respectively.

### Cell Preparation

2×10⁴ cells/ml IL-4R⁺ murine CTLL-2 cells were cultured in 20 ml of 10% Rat T-stim media (Becton-Dickinson, UK) in R10 media at 37° C., 5% CO₂ for 7 days. CTLL-2 cells are dependent on IL-2 and by day 7 most of the IL-2 is depleted from the media, thus the cells are now starved. Prior to the assay, the cells are harvested and washed three times in unsupplemented R10 by centrifugation at 300×g for 5 mins to remove any remaining IL-2.

### Cell Proliferation Assay Protocol

The following assay was carried out following manufacturer's instructions for the CellTiter-Glo® Luminescent Cell Viability Assay (Promega, US). Briefly:

The assay was carried out in opaque white flat-bottomed 96 well plates (Nunc, Denmark)

The following was added to each test well of the plate:

5×10³ murine CTLL-2 cells in 50 μl of R10 media
A range (1 nM-0.001 pM) of mIL-4-TCR fusion or recombinant murine IL-4 (Peprotech, UK) in 50 μl of R10 media

The following control wells were included:

100 μl of R10 media alone
5×10³ murine CTLL-2 cells in 100 μl of R10 media
5×10³ murine CTLL-2 cells in 50 μl of R10 media with 200 U/ml Pro-L (Chiron, The Netherlands) in 50 ul of R10 media

The test and control wells were then incubated at 37° C., 5% CO₂ for 24 hours

After incubation, 100 μl of the pre-mixed CellTiterGlo® reagent was added to each well. The plate was then placed on an orbital shaker for 2 minutes to induce cell lysis. The plate was then allowed to incubate for 10 minutes at room temperature. Finally, the luminescence from each well was read on the Wallac Victor 2 (Perkin Elmer, US) luminometer using a measurement time of 1.0 second per well.

### Results

As shown in FIG. 17 the proliferative response of the CTLL-2 cells to the mIL-4-TCR fusion was very similar to that induced by the recombinant murine IL-4. (mIL-4-TCR fusion EC50=approximately 1 pM, recombinant mIL-4 EC50=approximately 1 pM) These data demonstrate that the biological activity of the murine IL-4 part of the mIL-4-TCR fusion is comparable to that of recombinant murine IL-4.

1. A T-cell receptor (TCR) having the property of binding to the LYLVCGERG-H-2K^{d} complex and comprising at least one TCR α chain variable domain and/or at least one TCR β chain variable domain CHARACTERISED IN THAT said TCR has a K_{D} for the said LYLVCGERG-H-2K^{d} complex of less than or equal to 1 μM and/or has an off-rate (k_{off}) for the LYLVCGERG-H-2K^{d} complex of 0.5 S⁻¹ or slower.

2. A TCR as claimed in claim 1 comprising both a TCR α chain variable domain and a TCR β chain variable domain.

3. A TCR as claimed in claim 1 which is an αα or ββ homodimer.

4. A T-cell receptor (TCR) as claimed in claim 1 wherein the said K_{D} and/or k_{off} is/are as measured by Surface Plasmon Resonance.

5. A TCR as claimed in claim 1 which is mutated relative to the wild-type murine TCRα chain variable region shown in SEQ ID No: 1 and/or β chain variable region shown in SEQ ID NO: 2 in at least one complementarity determining region.

6. A TCR as claimed in claim 1 which is mutated relative to the wild-type murine TCRα chain variable region shown in SEQ ID No: 1 and/or β chain variable region shown in SEQ ID NO: 2 in at least one variable domain framework region thereof.

7. A TCR as claimed in claim 1 wherein one or more of alpha chain variable region amino acids 26Y, 27K, 28T, 29S, 30I, 31T, 32A, 95M, 97Y and 98K using the numbering shown in SEQ ID NO: 1 is/are mutated.

8. A TCR as claimed in claim 1 wherein one or more of beta chain variable region amino acids 51I, 52T, 53E, 54N, 55D, 82A, 83Q, 96I, 98D, 99R, 103T, 104L and 105Y using the numbering shown in SEQ ID NO: 2 is/are mutated.

9. A TCR as claimed in claim 1 comprising one or more of alpha chain variable region amino acids 26F, 27S, 28S, 28R, 28G, 28W, 28H, 29P, 29L, 29M, 29W, 30M, 30G, 30V, 30F, 31I, 31V, 31W, 32N, 32T, 95L, 95 F, 97W, 98I, 98V, 98R, 98M or 98Q using the numbering shown in SEQ ID NO: 1.

10. A TCR as claimed in claim 1 comprising one or more of beta chain variable region amino acids 51L, 52L, 52D, 52A, 52N, 53S, 53A, 53L, 53R, 53M, 53P, 53T, 54G, 54D, 54E, 54V, 54S, 54R, 54H, 54T, 55H, 55T, 82T, 82P, 82V, 83H, 96K, 96R, 96N, 96V, 96F, 98K, 98E, 98R, 98L, 99N, 99D, 99E, 99F, 99H, 103K, 103R, 104Y, 105F or 105I using the numbering shown in SEQ ID NO: 2.

11. A TCR as claimed in claim 1 comprising one of the alpha chain variable region amino acid sequences shown in (SEQ ID Nos: 11 to 25).

12. A TCR as claimed in claim 1 comprising one of the beta chain variable region amino acid sequences shown in (SEQ ID Nos: 26 to 55).

13. A TCR as claimed in claim 2 comprising an alpha and beta chain variable region pairing selected from the group consisting of
SEQ ID NO:11 and SEQ ID NO:2;
SEQ ID NO:12 and SEQ ID NO:2;
SEQ ID NO:13 and SEQ ID NO:2;
SEQ ID NO:14 and SEQ ID NO:2;
SEQ ID NO:15 and SEQ ID NO:2;
SEQ ID NO:16 and SEQ ID NO:2;
SEQ ID NO:17 and SEQ ID NO:2;
SEQ ID NO:18 and SEQ ID NO:2;
SEQ ID NO:19 and SEQ ID NO:2;
SEQ ID NO:20 and SEQ ID NO:2;
SEQ ID NO:21 and SEQ ID NO:2;
SEQ ID NO:22 and SEQ ID NO:2;
SEQ ID NO:23 and SEQ ID NO:2;
SEQ ID NO:24 and SEQ ID NO:2;
SEQ ID NO:25 and SEQ ID NO:2;
SEQ ID NO:11 and SEQ ID NO:26;
SEQ ID NO:11 and SEQ ID NO:27;
SEQ ID NO:11 and SEQ ID NO:28;
SEQ ID NO:11 and SEQ ID NO:29;
SEQ ID NO:11 and SEQ ID NO:30;
SEQ ID NO:11 and SEQ ID NO:31;
SEQ ID NO:11 and SEQ ID NO:32;
SEQ ID NO:11 and SEQ ID NO:33;
SEQ ID NO:11 and SEQ ID NO:34;
SEQ ID NO:11 and SEQ ID NO:35;
SEQ ID NO:11 and SEQ ID NO:36;
SEQ ID NO:11 and SEQ ID NO:37;
SEQ ID NO:11 and SEQ ID NO:38;
SEQ ID NO:11 and SEQ ID NO:39;
SEQ ID NO:11 and SEQ ID NO:40;
SEQ ID NO:11 and SEQ ID NO:41;
SEQ ID NO:11 and SEQ ID NO:42;
SEQ ID NO:11 and SEQ ID NO:43;
SEQ ID NO:11 and SEQ ID NO:44;
SEQ ID NO:11 and SEQ ID NO:45;
SEQ ID NO:11 and SEQ ID NO:46;
SEQ ID NO:11 and SEQ ID NO:47;
SEQ ID NO:11 and SEQ ID NO:48;
SEQ ID NO:11 and SEQ ID NO:49;
SEQ ID NO:11 and SEQ ID NO:50;
SEQ ID NO:11 and SEQ ID NO:51;
SEQ ID NO:11 and SEQ ID NO:52;
SEQ ID NO:11 and SEQ ID NO:53;
SEQ ID NO:11 and SEQ ID NO:54;
SEQ ID NO:11 and SEQ ID NO:55;
SEQ ID NO:24 and SEQ ID NO:40;
SEQ ID NO:24 and SEQ ID NO:45; and
SEQ ID NO:20 and SEQ ID NO:52.

14. A TCR as claimed in claim 1 comprising the alpha chain variable region amino acid sequence shown in SEQ ID NO: 20 and the beta chain variable region amino acid sequence shown in SEQ ID NO: 52.

15. A TCR as claimed in claim 1 further comprising a TCR alpha chain constant domain sequence selected from the group consisting of:
the murine alpha chain constant domain amino acid sequence shown in SEQ ID NO: 56, and
the human alpha chain constant domain amino acid sequence shown in SEQ ID NO: 66.

16. A TCR as claimed in claim 1 further comprising a TCR beta chain constant domain sequence selected from the group consisting of:
the murine beta chain amino acid constant domain sequence shown in SEQ ID NO: 57,
the human beta chain constant domain amino acid sequence shown in SEQ ID NO: 67, and
the human beta chain constant domain amino acid sequence shown in SEQ ID NO: 68.

17. A TCR as claimed in claim 1 which is a dimeric T cell receptor (dTCR) or a single chain T cell receptor (scTCR).

18. A TCR as claimed in claim 1 which is a scTCR comprising
a first segment constituted by an amino acid sequence corresponding to a TCR α chain variable region sequence,
a second segment constituted by an amino acid sequence corresponding to a TCR β chain variable region sequence fused to the N terminus of an amino acid sequence corresponding to a TCR β chain constant domain extracellular sequence, and
a linker sequence linking the C terminus of the first segment to the N terminus of the second segment.

19. A TCR as claimed in claim 1 which is an scTCR comprising
a first segment constituted by an amino acid sequence corresponding to a TCR β chain variable region sequence,
a second segment constituted by an amino acid sequence corresponding to a TCR α chain variable region sequence fused to the N terminus of an amino acid sequence corresponding to a TCR α chain constant domain extracellular sequence, and
a linker sequence linking the C terminus of the first segment to the N terminus of the second segment.

20. A scTCR as claimed in claim 18, wherein the said first and second segments are linked by a disulfide bond between a pair of cysteine residues substituted for amino acid residues selected from the group consisting of:
Thr 48 of the murine alpha chain extracellular constant domain using the numbering of SEQ ID NO: 56 and Ser 57 of the murine TCR beta chain extracellular constant domain using the numbering of SEQ ID NO: 57, and
Thr 48 of the human TCR alpha chain extracellular constant domain using the numbering of SEQ ID NO: 66 and Ser 57 of the TCR beta chain extracellular constant domain using the numbering of SEQ ID NO: 67 or SEQ ID NO: 68.

21. An scTCR as claimed in claim 1 wherein the linker sequence links the C terminus of the first segment to the N terminus of the second segment.

22. A scTCR as claimed in claim 1 wherein the linker sequence has the formula -PGGG-(SGGGG)₅-P- shown in SEQ ID NO: 63 or -PGGG-(SGGGG)₆-P- shown in SEQ ID NO: 64 wherein P is proline, G is glycine and S is serine.

23. A scTCR as claimed in claim 1 wherein the TCR chain variable region sequences in the first and second segments correspond to the functional variable domains of a first TCR, and the TCR α or TCR β chain constant region extracellular sequence present in the second segment corresponds to that of a second TCR, the first and second TCRs being from different species.

24. A TCR as claimed in claim 1 which is a dTCR comprising
a first polypeptide wherein a sequence corresponding to a TCR α chain variable region sequence is fused to the N terminus of a sequence corresponding to a TCR α chain constant domain extracellular sequence, and
a second polypeptide wherein a sequence corresponding to a TCR β chain variable region sequence fused to the N terminus a sequence corresponding to a TCR β chain constant domain extracellular sequence,
the first and second polypeptides being linked by a disulfide bond between a pair of cysteine residues substituted for amino acid residues selected from the group consisting of:
Thr 48 of the murine alpha chain extracellular constant domain using the numbering of SEQ ID NO: 56 and Ser 57 of the murine TCR beta chain extracellular constant domain using the numbering of SEQ ID NO: 57, and
Thr 48 of the human TCR alpha chain extracellular constant domain using the numbering of SEQ ID NO: 66 and Ser 57 of the TCR beta chain extracellular constant domain using the numbering of SEQ ID NO: 67 or SEQ ID NO: 68.

25. A dTCR as claimed in claim 24 wherein the TCR chain variable region sequences in the first and second polypeptides correspond to the functional variable domains of a first TCR, and the TCR chain constant region extracellular sequences present in the first and second polypeptide correspond to those of a second TCR, the first and second TCRs being from different species.

26. A dTCR as claimed in claim 1 comprising the TCR α chain sequence shown in SEQ ID NO: 60 and the TCR β chain sequence shown in SEQ ID NO: 61.

27. A TCR as claimed in claim 17 wherein the dTCR or scTCR includes an interchain disulfide bond between residues corresponding to those linked by an interchain disulfide bond in native TCRs.

28. A TCR as claimed in claim 17 wherein the dTCR or scTCR does not contain a sequence corresponding to transmembrane or cytoplasmic sequences of native TCRs.

29. A TCR as claimed in claim 1 wherein the TCR is associated with at least one polyalkylene glycol chain(s).

30. A TCR as claimed in claim 1 further comprising a reactive cysteine at the C-terminal or N-terminal of the TCR α chain or TCR β chain.

31. A TCR as claimed in claim 1 associated with a therapeutic agent or detectable moiety.

32. A TCR as claimed in claim 31 wherein the TCR is covalently linked to a therapeutic agent or detectable moiety.

33. A TCR as claimed in claim 32 wherein the therapeutic agent or detectable moiety is covalently linked to the N-terminus of one or both TCR chains.

34. A TCR as claimed in claim 31 associated with a therapeutic agent which is an immune effector molecule.

35. A TCR as claimed in claim 35 wherein the immune effector molecule is a cytokine.

36. A TCR as claimed in claim 35 wherein the immune effector molecule is IL-4, IL-10 or IL-13.

37. A TCR as claimed in claim 31 wherein the therapeutic agent is a radionuclide.

38. A multivalent TCR complex comprising at least two TCRs as claimed in claim 1.

39. A multivalent TCR complex comprising at least two TCRs as claimed in claim 1 linked by a non-peptidic polymer chain or a peptidic linker sequence.

40. A TCR complex as claimed in claim 39 wherein the non-peptidic polymer chain or peptidic linker sequence extends between amino acid residues of each TCR which are not located in a variable region sequence of the TCR.

41. A multivalent TCR complex comprising at least two TCRs as claimed in claim 1 wherein at least one of said TCRs is associated with a therapeutic agent.

42. A cell harbouring an expression vector comprising nucleic acid encoding a TCR as defined in claim 1.

43. A pharmaceutical composition comprising a TCR or a multivalent TCR complex as claimed in claim 1, together with a pharmaceutically acceptable carrier.

44. A method of assessing the efficacy of a putative anti-diabetic agent in a mammalian model of diabetes comprising:
treating one or a plurality of the said model mammals with a beta cell specific TCR associated with the said agent, said TCR having a K_{D} for the said LYLVCGERG-H-2K^{d} complex of less than or equal to 1 μM and/or has an off-rate (k_{off}) for the LYLVCGERG-H-2K^{d} complex of 0.5 S⁻¹ or slower,
monitoring the effect of such treatment on the treated mammals,
comparing the said effect with
(i) the null effect in one or more untreated control model mammals; and/or
(ii) the effect of treatment of one or more model mammals with the said agent in a form not associated with the said TCR; and/or
(iii) the effect of treatment of one or more model mammals with a different anti-diabetic or putative anti-diabetic agent in a form associated or not associated with the said TCR and/or
(iv) the effect of treatment of one or more model mammals with the said agent in a form associated with an irrelevant TCR.

45. A method of assessing the efficacy of a putative anti-diabetic agent in a murine model of diabetes comprising:
treating one or a plurality of the said model mice with a murine insulin specific TCR associated with the said agent, said TCR having a K_{D} for the said LYLVCGERG-H-2K^{d} complex of less than or equal to 1 μM and/or has an off-rate (k_{off}) for the LYLVCGERG-H-2K^{d} complex of 0.5 S⁻¹ or slower,
monitoring the effect of such treatment on the treated mice,
comparing the said effect with
(i) the null effect in one or more untreated control model mice; and/or
(ii) the effect of treatment of one or more model mice with the said agent in a form not associated with the said TCR; and/or
(iii) the effect of treatment of one or more model mice with the a different anti-diabetic or putative anti-diabetic agent in a form associated or not associated with the said TCR and/or
(iv) the effect of treatment of one or more model mice with the said agent in a form associated with an irrelevant TCR.

46. A method of assessing the mass of islet cells present in a mammal, said method comprising.
(i) contacting islet cells of the mammal with an islet cell specific TCR associated with a detectable moiety, said TCR having a K_{D} for the said LYLVCGERG-H-2K^{d} complex of less than or equal to 1 μM and/or has an off-rate (k_{off}) for the LYLVCGERG-H-2K^{d} complex of 0.5 S⁻¹ or slower.
(ii) detecting a signal from the islet cell-bound TCRs associated with a detectable label, and
(iii) using said signal to estimate the mass of islet cells present.

47. A method of assessing the mass of islet cells present in a mouse, said method comprising.
(i) contacting islet cells of the mammal with a murine insulin specific TCR associated with a detectable moiety, said TCR having a K_{D} for the said LYLVCGERG-H-2K^{d} complex of less than or equal to 1 μM and/or has an off-rate (k_{off}) for the LYLVCGERG-H-2K^{d} complex of 0.5 S⁻¹ or slower,
(ii) detecting a signal from the islet cell-bound TCRs associated with a detectable label, and
(iii) using said signal to estimate the mass of islet cells present.

48. A method as claimed in claim 44 wherein the non-irrelevant TCR is a TCR having the property of binding to the LYLVCGERG-H-2K^{d} complex and comprising at least one TCR α chain variable domain and/or at least one TCR β chain variable domain CHARACTERISED IN THAT said TCR has a K_{D} for the said LYLVCGERG-H-2K^{d} complex of less than or equal to 1 μM and/or has an off-rate (k_{off}) for the LYLVCGERG-H-2K^{d} complex of 0.5 S⁻¹ or slower.
